(19) United States
(12) Patent Application Publication
(10) Pub. No.: US 2003/0129583 A1
(43) Pub. Date: Jul. 10, 2003

(54) Therapy of stealth virus associated cancers and other conditions using light

(76) Inventor: William John Martin, South Pasadena, CA (US)

Correspondence Address: W. John Martin, 1634 Spruce St., South Pasadena, CA, 91030, US

(21) Appl. No.: 10/044,683

(22) Filed: Jan. 8, 2002

(51) Int. Cl. C12Q 1/70; G01N 33/53; G01N 33/537; G01N 33/543; C12N 7/00; C12N 7/01; C12N 5/00; C12N 5/02

(52) U.S. Cl. 435/5; 435/7.1; 435/7.92; 435/7.91; 435/325; 435/235.1

(57) ABSTRACT

A method of causing irreversible damage to stealth virus infected cells, based on light induced toxic photosensitivity of abnormal, aggregated, intracellular materials present in the infected cells. The method of the present application specifically relates to the culturing of stealth viruses from an infected subject and determining the presence in the stealth virus infected culture of auto-fluorescent material. Auto-fluorescent material can also be found in cultures of bacteria that are infected with the patient's stealth virus. The cultured stealth virus infected cells are to be further tested for susceptibility to the cellular destructive effect of light therapy given at various wavelengths that are not injurious to normal uninfected cells. The presence of auto-fluorescent material in the stealth virus culture and the demonstration that exposure to light can cause damage to stealth virus infected cells, form the basis of therapy for stealth virus infected patients and animals. The therapy comprises exposing infected cells within a patient to light at the wavelengths that were shown to cause damage to cultured virus infected cells. Light mediated therapy is especially applicable as therapy for stealth virus associated cancers in which the stealth virus isolated from the cancer patient can be shown to induce the formation of auto-fluorescent material.

### CLASS

424 DRUG, BIO-AFFECTING AND BODY TREATING COMPOSITIONS

### CROSS REFERENCE TO RELATED APPLICATIONS

United States Patents

U.S. Pat. No. 5,985,546 Stealth virus detection in the chronic fatigue syndrome William John Martin

U.S. Pat. No. 5,891,468 Stealth virus detection in the chronic fatigue syndrome William John Martin

U.S. Pat. No. 5,753,488 Isolated stealth viruses and related vaccines William John Martin

U.S. Pat. No. 5,703,221 Stealth virus nucleic acids and related methods William John Martin

U.S. Pat. No. 5,079,262, Method of Detection and Treatment of Malignant and Non-Malignant Lesions

Utilizing 5-Aminolevulinic Acid. James C. Kennedy et al. U.S. Pat. No. 6,323,012 “Method for treating viral infections”, awarded to Ben-Hur, et al.

PCT (Patent Cooperation Treaty)

WO 92/20797 Stealth virus detection in the chronic fatigue syndrome

WO 99/34019 Stealth virus nucleic acids and related methods

WO 99/60101 Stealth Viruses and Related Vaccines

### REFERENCES TO PUBLISHED ARTICLES

Stealth Viruses:

1 Martin W J Chronic fatigue syndrome among physicians. A potential result of occupational exposure to stealth viruses. Explore 2001; 10 (5): 7-10.

2 Martin W J. Stealth Viruses. Explore 2001; 10 (4): 17-19.

3 Durie G M, Collins R. Martin W J. Positive stealth virus cultures in multiple myeloma. A possible explanation for neuropsychiatric co-morbidity. Presented at the Am. Soc. Hematology annual meeting October 2000.

4 Martin W J. Chemokine receptor-related genetic sequences in an African green monkey simian cytomegalovirus-derived stealth virus. Exp Mol Pathol. 2000; 69:10-6.

5 Martin W J. and Anderson D. Stealth virus epidemic in the Mohave Valley: severe vacuolating encephalopathy in a child presenting with a behavioral disorder. Exp Mol Pathol. 1999; 66:19-30.

6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin W J. Bacteria-related sequences in a simian cytomegalovirus-derived stealth virus culture. Exp Mol Pathol. 1999; 66:8-14.

8 Martin W J. Stealth adaptation of an African green monkey simian cytomegalovirus. Exp Mol Pathol. 1999; 66:3-7.

9 Martin W J. Cellular sequences in stealth viruses. Pathobiology 1998; 66:53-8.

10 Martin W J. Detection of RNA sequences in cultures of a stealth virus isolated from the cerebrospinal fluid of a health care worker with chronic fatigue syndrome. Case report. Pathobiology. 1997; 65:57-60.

11 Martin W J. and Anderson D. Stealth virus epidemic in the Mohave Valley. I. Initial report of virus isolation. Pathobiology. 1997; 65:51-6.

12 Martin W J. Simian cytomegalovirus-related stealth virus isolated from the cerebrospinal fluid of a patient with bipolar psychosis and acute encephalopathy. Pathobiology. 1996; 64:64-6.

13 Martin W J. Stealth viral encephalopathy: report of a fatal case complicated by cerebral vasculitis. Pathobiology. 1996; 64:59-63.

14 Martin W J. Genetic instability and fragmentation of a stealth viral genome. Pathobiology. 1996; 64:9-17.

15 Martin W J. Severe stealth virus encephalopathy following chronic-fatigue-syndrome-like illness: clinical and histopathological features. Pathobiology. 1996; 64:1-8.

16 Martin W J. Stealth virus isolated from an autistic child. J Autism Dev Disord. 1995; 25:223-4.

17 Gollard R P, Mayr A., Rice D A, Martin W J. Herpesvirus-related sequences in salivary gland tumors. J Exp Clin Cancer Res., 1996; 15: 1-4.

18 Martin W J. and Glass R T. Acute encephalopathy induced in cats with a stealth virus isolated from a patient with chronic fatigue syndrome. Pathobiology. 1995; 63:115-8.

19 Martin W J, et al. African green monkey origin of the a typical cytopathic ‘stealth virus’ isolated from a patient with chronic fatigue syndrome. Clin Diag Virol 1995: 4: 93-103.

20 Martin W J. Stealth viruses as neuropathogens. CAP Today. 1994; 8: 67-70.

21 Martin W J. et al. Cytomegalovirus-related sequence in an a typical cytopathic virus repeatedly isolated from a patient with chronic fatigue syndrome. Am J Pathol. 1994; 145: 440-51.

### OTHER CITED REFERENCES

Aust G J. Endocrinol 2001; 170:513-20.

Dyer R B. and McMurray C T. Nat Genet 2001; 29: 270-8.

Haghnegahdar H et al. J Leukoc Biol 2000; 67: 53-62.

Ismail A. et al. Heart Dis 1999; 1: 233-40.

Kaneko I. et al. Neuroscience 2001; 104:1003-11.

Kellner-Weibel G. et al. Arterioscler Thromb Vasc Biol 1999; 19: 1891-8

Moore B B. Am J Pathol. 1999; 154: 1503-12.

Pati S. J Virol. 2001; 75: 8660-73.

Reece P H J Laryngol Otol. 1999; 113: 174-6

Robinson S. Neurosurgery. 2001; 48: 864-73.

Schweitzer V G. Lasers Surg Med. 2001; 29: 305-313.

Sibata C H et al. Expert Opin Pharmacother. 2001; 2: 917-27

Vsevolodov N “Biomolecular Electronics An Introduction via Photosensitive Proteins” Birkhauser Press, Boston 1998.

Wadsworth J D et al. Lancet 2001; 358:171-80.

Yang S K. Gastroenterology. 1997; 113: 1214-23.

### STATEMENT REGARDING FEDERALLY SPONSORED RESEARCH OR DEVELOPMENT

No Federal funding was received in support of the research covered in this patent application.

### REFERENCE TO SEQUENCE LISTING, A TABLE, OR A COMPUTER PROGRAM LISTING COMPACT DISK APPENDIX

None provided.

### BACKGROUND OF THE INVENTION

The present invention relates to therapy of virus infections, in which the virus belongs to a group of a typically structured, non-inflammation-inducing viruses, for which the inventor has coined the term stealth viruses. The patent application relates particularly to the therapy of cancers in which the malignant cells are infected with a stealth virus. Methods for the detection and characterization of stealth viruses are covered in U.S. Pat. Nos. 5,985,546; 5,891,468; 5,753,488; and 5,703,221. Although initially identified in association with neuropsychiatric illnesses, including the chronic fatigue syndrome, stealth viruses can also commonly be cultured from cancer patients, and can be detected within tumor tissues. (Stealth virus-related references are listed in this application and are numbered 1-21. All cited patents and entire list of stealth virus publications are incorporated herein by reference).

The basis of the present invention is the discovery that certain stealth viruses lead to marked intracellular accumulations of a diverse range of particulate materials, some of which are photo-(light) sensitive. Light is the visible portion of the extremely broad spectrum of electromagnetic radiation, that also includes non-visible energies such as gamma rays, X-rays, microwaves, radio-waves, and both ultraviolet and infrared radiations. Light is best understood in terms of energy packets termed photons that travel through space in an oscillating sine-wave form. The energy of each photon is determined by the frequency of the fluctuating wave, which is typically measured as a wavelength of each full oscillation. Visible white light comprises a mixed range of colors that extend from the relatively short wavelengths around 400×10⁻⁹ meters, (400 nanometers) for purple light to the longer wavelengths of around 600-700 nanometers (nm) for the varying shades of red light. Portions of the light spectrum of white light are either absorbed or reflected by all but fully transparent objects. The perceived color of an object is that of the reflected light waves. For the vast majority of objects, the energy of the absorbed light is dissipated as an increase in the motion of the individual molecules that comprise the object. This results in a rise in the temperature of the object. For certain compounds, however, parts of the energy of the incoming light can cause an orbital shift in the outer electrons surrounding individual atoms. As these “excited” electrons return towards their previous energy levels, they can release photons and become a source of re-emitted light. The electrons can also become involved in mediating certain chemical reactions and ion transfers that are dependent upon, and driven by, the heightened energy state.

The term “photosensitive” specifically refers to the ability of certain materials to absorb energy from light, at one or more particular wavelengths, and to subsequently emit some of the absorbed energy as light, usually at a longer wavelength than that causing the photo-excitation. This light absorption/re-emission process is termed auto-fluorescence. It is generally known that unregulated and/or excessive light induced fluorescence occurring within a cell can have detrimental effects on cell vitality and can lead to cell death. This phenomenon is the basis for a suggested form of cancer therapy known as photodynamic therapy (Sibata C H et al. Expert Opin Pharmacother. 2001; 2: 917-27). In conventional photodynamic therapy a photosensitive substance, or its metabolic precursor, is systemically administered and allowed to accumulate within cancer cells. The photosensitive substance is then excited with light, usually administered as a laser beam corresponding to the maximum excitatory wavelength of the administered photosensitizing agent. The most commonly suggested photosensitizing agent is 5-aminolevulinic acid (ALA). This is a precursor of protoporphyrin IX which is an auto-fluorescent molecule involved in haem synthesis. The potential use of ALA to photosensitize tumor cells is covered by U.S. Pat. No. 5,079,262, entitled “Method of Detection and Treatment of Malignant and Non-Malignant Lesions Utilizing 5-Aminolevulinic Acid,” issued to James C. Kennedy et al. on Jan. 7, 1992. This method has yet to find widespread use in human or animal cancer therapy. The potential use of ALA was recently also advocated for the photodynamic therapy of virus infected cells in U.S. Pat. No. 6,323,012 “Method for treating viral infections”, awarded to Ben-Hur, et al. on Nov. 27, 2001. A directly photosensitive compound in limited clinical use for superficial cancers, or for cancers that can be accessed by fiberoptic laser light, is an analog of protoporphyrin IX called porfimer sodium (Schweitzer V G. Lasers Surg Med. 2001; 29: 305-313).

### BRIEF DESCRIPTION OF THE DRAWINGS (FIGURES)

The accompanying drawings, which are incorporated in and constitute a part of the specification, illustrate the various findings described in this application. They serve to explain the foundations and the principles of the invention. The drawings (Figures) are best view as Landscape prints.

FIG. 1 shows the low power (10× objective) microscopic appearance of cultured normal MRC-5 human fibroblasts. The culture consists of a flat monolayer of closely packed, interdigitating, spindle shaped, non-pigmented cells, which have a rather bland appearance. This type of appearance persists throughout a negative culture, except for some minor retraction and cell shrinkage seen during the second and third week of culture.

FIG. 2 shows the low power appearance of a clearly positive stealth virus culture. The changes that are shown occurred within a week of adding a frozen-thawed extract of mononuclear cells that were isolated from the blood of a stealth virus positive patient. During the first few days of culture many of the cells throughout the culture began to change from a long, thin, spindle shape, to a wider, more rounded shape. There was an increase in the amount of cytoplasm, which acquired a foamy, somewhat vacuolated and retractile appearance. Many of the cells started to disappear from the culture, while the remaining cells tending to adhere to, and even to fuse with each other.

FIG. 3 shows the higher power (20× objective) view of the clearly positive culture that was shown in FIG. 2. The cells tend to fuse with each other to form varying sized syncytia. Additionally, larger cell clusters can be readily seen.

FIG. 4 shows a large, darkly pigmented, cell cluster. The pigmentation extends throughout the cell cluster, except for a narrow rim of outside cells. Varying shades of pigmented cell clusters are commonly seen in different stealth virus cultures. Many of the individual cells and small cell clusters can also acquire a pigmented appearance, generally consisting of fine to coarse granular material.

FIG. 5 shows the compacting and localization that can occur to the pigmented material that develops within a cell cluster. The localization of the pigment is especially pronounced in cultures that are left unfed for extended periods of time. The pigment can actually be extruded from the clusters.

FIG. 6. Photomicrographs showing the growth of relatively normal appearing cells from around the edges of cell clusters. Especially if the culture medium is not replaced, many of the cell clusters in a culture will begin to show evidence of cell proliferation. This can be seen as the emergence of relatively normal appearing cells from the periphery of the clusters. The bottom photograph is from the culture shortly after replacement of the tissue culture medium. After several days, the clusters shown in this photo have become ringed with freshly dividing cells (upper photograph).

FIG. 7. This photomicrograph is from a stealth virus culture. It shows a long thread/tube-like structure emerging from a relatively small cell cluster. The structure extends past several additional cell clusters, 4 of which are shown in this photo. Note the pigment within the clusters and the evidence for cell outgrowths from two of the clusters.

FIG. 8. Continuation of the same thread/tube-like structure as seen in FIG. 7. These structures can be seen growing over a several day period.

FIG. 9. A low power photomicrograph showing a long thread/tube-like structure emerging from small a cell cluster. The rest of the culture shown in this photograph has largely been repaired and now consists of relatively normal appearing cells, not unlike the appearance shown in FIG. 1.

FIG. 10. A higher power photomicrograph of the same structure shown in FIG. 9. It more clearly shows the origin of the structure from the small residual cell cluster.

FIG. 11. A tubular appearing structure emerging from a lightly pigmented cell cluster present in a stealth virus culture.

FIG. 12. A similar structure as shown in FIG. 11 emerging from the center of a degenerating cell cluster from a long established stealth virus culture.

FIG. 13. A complex pattern of multiple ribbons and tube-like structures developing from the cell cluster shown in the center of the photograph. This cluster can be contrasted with the cluster in the upper right hand corner that does not have any protruding structures.

FIG. 14. A higher magnification of cell cluster shown in FIG. 13 demonstrating the complex uneven staining pattern of the ribbons, tube-like structures and pigmented deposits.

FIG. 15. A high power view of a large complex ribbon-like structure that was laying free in a stealth virus culture. Fine intricate and irregular details can be seen within the ribbon-like structure on close examination.

FIG. 16. A nearly transparent, large diamond shaped, crystal-like structure that is positioned at the right side of the junction of two large cell clusters present in a stealth virus culture.

FIG. 17. A dark field photomicrograph of an irregularly shaped, complex and variably pigmented, plate-like structure seen under low power, in a stealth virus culture. Small micro-tubular elements can be seen both inside and emerging from the structure. The particular structure was seen in the culture from the patient with Dercum's disease that is referred to in the specification.

FIG. 18. A complex mixture of particulate materials and fine needle like structures seen by dark field microscopy in the supernatant of a stealth virus culture. Most of the material seen in this photograph had a significant lipid component as shown by its partitioning into a chloroform phase, when this lipid solvent was added to the medium.

FIG. 19. Dark field appearance of an extensive collection of multiple needle-like structures as seen under dark field illumination. The culture was derived from the patient known to be infected with an SCMV-derived stealth virus. The sets of two parallel sides that can be seen generally represent the thicker edges that are seen at the sides of the needles and or curved plates. Other forms of cholesterol-like crystals, not seen in this particular photo, include flat, notched plates. The differing appearances are thought to reflect whether the cholesterol is derived from a cholesterol ester (which tends to form needle shaped structures) or from free cholesterol (which tends to form flat, plate-like structures). The needles are not pure cholesterol, however, since they are only partially soluble in chloroform.

FIG. 20. Electron micrograph of a cell derived from a stealth virus culture. In addition to numerous lipid-filled vacuoles, the cell shows numerous, varying sized, deposits of fine and coarse granular materials throughout the cytoplasm. As is typical for many stealth virus cultures, no actual viral particles can be seen.

FIG. 21. Low magnification electron micrograph of a cell from a different stealth virus culture showing an extraordinarily extensive collection of coarsely granular materials that extends in a broad band throughout much of the cytoplasm. The outline of the nucleus can be seen above the lower left half of the inclusion. Several large vacuoles are also seen within the cell.

FIG. 22. A higher magnification electron micrograph of a cell that contains a similar appearing coarse granular material as that shown in FIG. 21.

FIG. 23. Electron micrograph showing lamellar-like bodies within the cytoplasm of a stealth virus infected cell. This particular culture was obtained from a child with a severe stealth virus encephalopathy.

FIG. 24. Numerous crystal-like structures present in the dried agar from a selected bacteria culture obtained from the stool sample of a stealth virus infected patient. A wide range of differing crystal-like and other types of structures can be produced in the bacterial cultures obtained from stealth virus infected patients.

FIG. 25. A complex branching thread-like structure seen in a culture of bacteria that had been exposed to a stealth virus cellular culture and subsequently plated onto a tryptic soy agar plate.

FIG. 26. A low electron micrograph of the brain biopsy obtained from the child with the positive stealth virus culture referred to FIG. 23. There is marked degeneration and clefting of the myelin sheaths, with a dropout of the axons. The cell itself, shows numerous vacuoles and disrupted mitochondria. The figure is presented primarily because it also shows an unusual appearing heart-shaped inclusion. This structure bears no resemblance to any known type of normal cellular structure. Nor does it correspond to any known microbial agent.

FIG. 27. A high power view of the abnormal appearing heart-shaped structure seen in FIG. 26.

FIG. 28. Several additional examples of the oddly shaped and unevenly staining structures that were seen within the cytoplasm of several cells examined from this stealth virus infected child. The extensive vacuolization of the particular cell shown is also apparent in this photograph.

FIG. 29. This photograph is also from the electron microscopic examination of the brain biopsy from the child. It shows yet another formation of thin strands of more darkly containing material, that enmesh multiple round paler staining entities.

FIG. 30. Another example of an unusual cellular inclusion seen in the brain biopsy from the stealth virus infected child. This particular electron micrograph shows two fingerprint-like bands of parallel fine tubular structures embedded in amorphous material. Note the fine helical structure just beneath the upper band of the tubular material. Again, there is no known normal cellular structure with this type of appearance.

FIG. 31. Electron micrograph of a different cell from the same brain biopsy. It also shows very fine helical structures adjacent to and larger inclusion that contains irregularly shaped, very densely staining material. The cell has an abundance of disorganized cellular filaments.

FIG. 32. Electron micrograph of a glial cell seen in the brain biopsy of a stealth virus infected patient. In addition to the several vacuoles, some of which have retained their lipid content in spite of the fixation process, the cell contains irregularly shaped bands of darkly staining material. This material can be seen above the myelinated axon, within and separating the neural filaments and in the upper left hand side of the cell.

FIG. 33. An abnormal cell identified in a fine needle aspirate of the submandibular gland of a stealth virus infected patient. The particular patient has experienced salivary gland enlargement. The cell showed similar extensive cellular disruption with several lipid vacuoles and both fine and coarse collection of granular materials. The large black structures outside of the cell are erythrocytes. This particular patient subsequently developed both melanoma and thyroid cancer.

FIG. 34. Regular phase contrast photomicrograph of a typical tube-like structure extending from a cell cluster and passing nearby another cell cluster. It was seen in a partially repaired stealth virus culture.

FIG. 35. The same structures as seen in FIG. 34 when examined under slightly reduced magnification for auto-fluorescence. The white color indicates the emission of light from the specimen. The tube-like structure and especially the edges of the cell clusters near the tube-like structure are auto-fluorescent. Patchy auto-fluorescence is also seen in a small cell cluster to the right of the photograph.

FIG. 36. Auto-fluorescence of a long thread-like structure and of the attached cell clusters obtained from a stealth virus culture.

FIG. 37. Red auto-fluorescence induced in a cell from a stealth virus. The cell actually comprised two cells that had fused together. The two nuclei were in close proximity in the lower half of the cell. The white color represents the emission of red light passed through a red filter that was activated by exposing the cell to green laser light (543 nm). The same cell also showed significant green auto-fluorescence evoked by a blue laser (488 nm), but this was less intense and less widely distributed than the red auto-fluorescence. The photograph was taken on Halloween

FIG. 38. This photograph is included to show the rather modest, and limited distribution of green auto-fluorescence that was excited from exposing a tube-like structure obtained from a stealth virus culture in blue laser light. The fluorescence is indicated by the white areas along the length of the tube-like structure.

FIG. 39. This photograph shows the much more intense red auto-fluorescence emitted from the same structure as shown in FIG. 38. The exposure was reduced to indicate that even with the red auto-fluorescence, the source of light was not evenly distributed along the length of the structure.

FIG. 40. Irregular red auto-fluorescence in a crystal-like formation present in the agar of a bacterial culture. The bacteria were selected from a stool culture of a stealth virus infected patient. The crystal-like structure measured approximately 1 mm.

FIG. 41. A brightly staining structure seen in a bacterial culture that was obtained by exposing normal bacteria to a positive stealth virus culture of a patient with breast cancer.

FIG. 42. Comparison of the red auto-fluorescence (left panel) and green auto-fluorescence (right panel) of a small crystal fragment obtained from a bacterial culture. This structure showed a more intense red than green auto-fluorescence.

FIG. 43. A pre-light therapy photograph of a cell cluster present in a recovering stealth virus culture. Pigmented materials from the cluster have been localized into two deposits labeled A and B. Between the deposits a tube-like inclusion can be seen passing through the cell cluster. The focus of the experiment was on region labeled C, from which several healthy appearing cells had emerged. Although not shown in this high power photograph, the cell cluster was amidst a large number of relatively normal appearing fibroblasts that had shown recovery from an earlier virus-induced CPE.

FIG. 44. The same cell cluster as shown in FIG. 43 after 20 minutes of continued exposure to bright white light from the microscope. Several of the cells previously emerging from region C had withdrawn to the cell cluster, leaving only a single cell emerging from this region of the cluster.

FIG. 45. The same cell cluster as shown in FIG. 43, after 60 minutes of continued exposure to bright white light from the microscope. The remaining cell emerging from region C is now seen only as a narrow strand of very thin cytoplasm.

FIG. 46. The same cell cluster as shown in FIG. 43 after 120 minutes of continued exposure to white light from the microscope. At this time point, the remaining cell strand seen after 60-minutes exposure, had disappeared. Note: the light exposure was discontinued at 120 minutes and the culture tube was placed back into the incubator to test for cell recovery.

FIG. 47. This low power photograph was taken 4 hours after the light exposure had been discontinued. It shows the very extensive damage that occurred to many of the cells in the vicinity of the cell cluster shown in the previous photographs. All of the area shown had been included in the light that had been shone onto the culture tube.

FIG. 48. A photograph showing the lack on any recovery of viable cells from region C of the same cell cluster exposed 6 days earlier to light. Most, if not all of the cells in the cell cluster appeared to be non-viable. The extensive cellular destruction that was seen in the surrounding areas had similarly persisted. The culture was observed for an additional two weeks without any signs of cell recovery.

### BRIEF SUMMARY OF THE INVENTION

The invention provides a novel approach to treating a stealth virus infected patient by causing irreversible damage to stealth virus infected cells using light. The method is based on the discovery that stealth virus infected cells can produce abnormal, aggregated, intracellular and extra-cellular materials, and that this material can be auto-fluorescent. Light activation of the auto-fluorescent material provides a means of inducing cell damage to the stealth virus infected cells. The method of the present application specifically relates to the culturing of stealth viruses from infected patients, and determining the presence in the stealth virus infected cultures of auto-fluorescent material. This material can also be sought in bacteria infected with the patients' stealth viruses. The infected cells can be further tested for their sensitivity to the cellular destructive effect of light therapy given at a wavelength shown to be active in inducing cellular auto-fluorescence. The detection of auto-fluorescent materials in the stealth virus culture from a patient forms the basis of exposing infected cells within the patient to light at selected wavelengths, for the specific purpose of killing the viral infected cells. This approach is particularly applicable to the therapy of stealth virus associated cancers, in which the stealth virus isolated from the cancer patient can be shown to induce the formation of auto-fluorescent materials. This method will have wider applications, including the destruction of stealth viruses infected cells found in many other disease states, in both humans and animals. It can also be applied to destroying suspected stealth virus infected cells in blood used for transfusion and in tissues used for transplantation. It can also be applied as a method to destroy stealth virus infected bacteria that are producing photosensitive materials.

### DETAILED DESCRIPTION OF THE INVENTION

The present invention provides a method to selectively damage stealth virus infected cells within a patient. The method is based on determining the presence of photosensitive materials in stealth virus infected cells, and in the extra-cellular materials produced in stealth virus cultures. These materials will auto-fluorescence when exposed to light. The relative excitation effects of light at various defined wavelengths can be determined using specific lasers and light sources of known wavelengths, and by the use of colored filters. The ability of white light, and light at various defined wavelengths, to damage stealth virus infected cells in vitro can be readily determined microscopically. Based on the finding of auto-fluorescent materials in stealth virus cultures, in vivo infected cells can be confidently exposed to tissue penetrating light at the selected wavelengths, as a means to induce in vivo damage of stealth virus infected cells. This method has therapeutic applications in the therapy of stealth virus infected human and animal subjects.

The immune system is essentially incapable of killing stealth virus infected cells. As detailed in a series of publications, (listed above as numbers 1-21), stealth viruses do not evoke an inflammatory, cell killing, immune reaction. It is imperative, therefore, to find alternative methods of destroying stealth virus infected cells. This is especially so in the case of stealth virus infections that occur in association with cancers. The inventor has stressed the potential capacity of stealth viruses to induce cancers in several of his publications (numbers: 3,6,11,17). He has shown that the potential mechanism for the oncogenicity (cancer causing) capacity of stealth viruses is through the assimilation of various cell growth controlling genes (oncogenes) into a stealth virus genome (6,9). He has used tissue culture based methods to test many cancer patients for stealth virus infections, finding clearly positive culture results in the majority of those tested. In contrast, among a group of individuals providing blood for routine blood transfusion, the occurrence of a positive culture was less than 10%. When specifically asked, cancer patients and their family members will commonly describe symptoms of fatigue, memory loss, personality change, arthralgia and muscle aches; all of which are consistent with a pervasive stealth virus infection affecting the entire family. The focus of this application is on a method of destroying stealth virus infected cells. It is based on the discovery that these cells can produce materials that can be toxic for cells when activated with light. While the most immediate clinical application is for therapy of stealth virus associated cancers, there are many additional clinical applications apparent to those familiar with the multi-system illnesses associated with stealth virus infections.

The inventor has specifically discovered that stealth virus infected cells will commonly accumulate a wide range of intracellular photosensitive (auto-fluorescent) materials, and that cells infected with these stealth viruses can be killed by photo-activation of the materials by exposure to light. The origins of the photosensitive materials may be either from viral damaged cellular lipo(glyco)proteins, or from abnormal protein and other products arising from mutated viral, cellular or bacterial genes incorporated into the genome of the replicating stealth virus. Both by morphological criteria, and by the diverse patterns seen in auto-fluorescence studies, it is apparent that differences exist in the types of auto-fluorescent materials present in different stealth virus cultures. This is consistent with the molecular heterogeneity that exists among different stealth viruses (9,14). The capacity to culture stealth viruses from patients allows for the detailed characterization of the photo-excitability of the materials present in the stealth virus cultures from different patients. It also allows for the in vitro testing of the cell damaging effects of exposing infected cell cultures to the specific wavelengths of light shown to induce maximum auto-fluorescence. The methods and procedures can be applied to the therapy of both human and animals.

As used herein, photosensitizing materials refer to molecules that can capture radiant energy from light and subsequently emit light, generally of a different and longer wavelength than that absorbed. This property is called auto-fluorescence and such molecules have also been termed chromophores. In nature, chromophores play an essential role in energy absorption from sunlight, and in the detection and interpretation of light rays, as in vision. There are two major categories of light absorbing materials, with an increasing number of additional types of auto-fluorescence being described in the literature. Chlorophylls are light absorbing molecules that are based on a porphyrin structure and function mainly in plants and certain bacteria to allow for photosynthetic production of carbohydrates. Rhodopsins are present throughout evolution and include light sensing receptors in unicellular organisms, as well as the light sensing molecules in the rods and cones of the retina of the eye. Rhodopsins contain a reactive retinal molecule attached to an opsin protein, which typically has the structure of a G-protein receptor. (Of interest, a prototype stealth virus was shown to have multiple divergent copies of a G-protein receptor (4). They are structurally more closely related to a G-protein chemokine receptor than to a rhodopsin molecule, but have yet to be tested for their photosensitivity).

The absorption spectrum of various rhodopsin and several other photosensitive complexes is heavily influenced by such factors as primary amino acid sequence, pH, ions, water of hydration, lipid environment, ultrasound, electromagnetic and radio-frequency fields, etc, (Vsevolodov N. Biomolecular Electronics: An Introduction via Photosensitive Proteins. Birkhauser, Boston 1998). It can typically be quite broad, responding to a varying extent to a wide spectrum of frequencies occurring in incoming white light. In the normal setting, the secondary light and accompanying free radicals and ions that results from light exposure of chromophores, are channeled into functional pathways of either energy acquisition or light detection. In a cell with an abnormal collection of photosensitive materials, the captured light energy is released in the form of unregulated secondary light radiation, along with toxic release of free radicals and/or the movement of ions (such as singlet-oxygen atoms). Free radicals can be particularly damaging to the lipids of cell membranes, while ion movements can drastically alter pH and the levels of intracellular calcium and other cations. Auto-fluorescence can, therefore, lead to cell damaging effects, including cell death.

As used herein stealth or stealth-adapted viruses refers to infectious agents that will induce a characteristic vacuolating cytopathic effect (CPE) in human and animal tissue culture cells using procedures described for the cultivation of stealth viruses. These procedures have been provided in various patents and publications relating to stealth viruses. Essentially, it is possible to demonstrate the presence of a stealth virus in peripheral blood or tissues of a stealth virus infected patient, by following tissue culture procedures that will allow for the expression of a stealth virus induced CPE. A suitable procedure is as follows: Mononuclear cells are separated from 8 ml of whole blood, collected in an acid citrate dextrose (ACD) blood vaccutainer tube using Ficoll Paque (Pharmacia, N.J.) density centrifugation. After washing the mononuclear cells in phosphate buffered saline, they are resuspended in 2 ml of serum free, X Vivo-15 medium (BioWhittaker Inc., Md.). The cells are aliquoted into two vials, each of which is stored frozen until testing. The supernatant and the cell pellet from a lightly centrifuged thawed vial are each added to culture test tubes containing MRC-5 human fibroblasts (BioWhittaker Inc., Md.), in 3 ml of serum free X Vivo-15 medium. The culture tubes are placed on a slowly rotating wheel (Cel-Gro, Lab Line, Medford Ill., 4 minutes per rotation) in a 36.50 incubator. The tubes are examined regularly using an inverted phase contrast microscope. The appearance, rate of progression and host range of the CPE caused by stealth-adapted viruses are quite dissimilar from those caused by any of the commonly encountered conventional human cytopathic viruses, including human herpes simplex viruses, cytomegalovirus, Epstein-Barr virus, varicella-zoster virus, human adenoviruses, measles virus, or enteroviruses. In the case of MRC-5 human fibroblast indicator cells, the normal spindle shaped, translucent, closely packed cells become enlarged, rounded and tend to fuse into small, and later into larger, three dimensional cell syncytia and clusters. The cellular cytoplasm displays a vacuolated, lipid-laden-like appearance. With time, and especially in larger cell clusters, an additional accumulation of yellow-brown to golden-black, fine and/or coarse pigmentation, can be readily seen within affected cells, and sometimes in the culture supernatant. Even more striking is the formation of long extra-cellular pigmented thread, ribbon and tube-like structures in many of the longer-term cultures. These unusual structures are not seen in cultures of conventional cytopathic viruses. Extensive fine debris-like lipid materials, and longer cholesterol-like needles may also be seen in some stealth viral cultures. The levels of measurable cholesterol can reach up to 10 mg/100 ml of culture medium, compared to undetectable levels (<1 mg/100 ml) in the original X Vivo-15 medium. Similarly, the triglyceride level can be over 20 mg/100 ml in some of the cultures, compared with 9 mg/100 ml in normal X Vivo-15 medium.

While there are major overall similarities between stealth virus cultures from different patients, there are also many subtle differences, especially in terms of the extent, coarseness, color and types of particulate intracellular and extra-cellular materials, and in the tendency to form smaller or larger cell syncytia and cell clusters. The rate of progression of the CPE can also differ between cultures, but in most cases can be clearly promoted by frequent (every 2-3 days) replacing of the tissue culture medium.

Whereas phase contrast microscopy is routinely used to view the viral CPE, dark field microscopy is the preferred method for viewing cholesterol-like crystals, and many of the other extra-cellular materials. When using dark field microscopy, it was apparent that side-entering white light could cause abnormal cells in some of the stealth virus cultures to emit light, as opposed to simply reflecting the incoming light. This property generally correlated with the appearance of pigmented materials within the cells. Many of the longer extra-cellular structures would also be unusually bright on dark field illumination, sometimes even showing distinct red, green and blue coloration. With regular bright light, occasional examples were observed when a group of cells, or an extra-cellular structure, would show a brief afterglow following turning off the microscope lamp. For these reasons, the cultured cells and other structures were harvested for viewing under a fluorescent microscope. When viewed with a mercury vapor lamp in a Zeiss 310 confocal microscope, many of the abnormal cells emitted readily detectable green and red fluorescence. Bright green and red fluorescence was also achieved using the microscope's argon (blue light at 488 nm) and helium/neon (green light at 543 nm) laser light sources. Red auto-fluorescence was not seen with normal uninfected MRC-5 fibroblasts, and only a very low level of green auto-fluorescence was seen with normal cells using the argon laser. Most of the extra-cellular structures also showed complex uneven patterns of light induced red, and generally to a lesser extent, green auto-fluorescence. Examples of culture-derived auto-fluorescence cells and extracellular materials are provided later in this specification.

In various tissue biopsies from stealth virus infected patients and from animals injected with stealth viruses, the inventor has repeatedly noted the presence of unusual aggregates of particulate materials. As reported in various publications, periodic acid Schiff (PAS) stain will commonly show varying sized inclusions in histological sections of tissues from stealth virus infected patients (13,15) and animals (18). Additional details on the nature of these inclusions have been provided by electron micrographic studies. In most cases, the materials do not show an orderly structure, although occasionally distinct patterns can be seen, such as fine wavy lines or spirals. These a typical structures are quite different from any known normal intracellular component. Very unusual inclusions, including complex aggregates of intracelluar materials, were seen, for example, in the electron micrographs of each of the brain biopsies obtained from three patients with a severe stealth virus encephalopathy that followed a chronic fatigue syndrome-like illness (15). Different types of abnormal structures were seen by electron microscopy in a more recent brain biopsy from a stealth virus infected child (5). Examples of some of the structures seen in two of these brain biopsies, and in a fine needle aspirate of the submandibular gland of a stealth virus culture positive patient, are provided later in this specification. A wide range of extraneous structures can also be commonly seen in electron micrographs of infected cells obtained from stealth virus cultures (11). Examples are also provided later in the specification.

An explanation for the formation of extraneous structures in infected tissues and in stealth virus cultures is provided by the finding that stealth viruses can assimilate and mutate various genes of both cellular (9) and bacterial (7) origins. Many proteins can exist either as free soluble molecules, or be incorporated into larger macromolecular structures through an assembly process in which similar and/or different proteins are linked together to form insoluble protein complexes. Reactive lipids and other compounds can help mediate protein cross-linkage. It is reasonable to assume that among the many products that could be synthesized by stealth virus incorporated genes, some will have the tendency to cross-link with one another, or with themselves. The formation of macromolecular insoluble structures might also be facilitated because of the presence of other compounds, such as reactive lipids, in stealth virus cultures. The diversity of proteins and other components made by different stealth viruses would explain the lack of uniformity of the appearance of the various macromolecular complexes seen in the stealth virus cultures from different patients. These structures show auto-fluorescence presumably because some of the incorporated proteins (or other components) are photo-reactive. Although photo-reactivity is not a common property of most normal proteins, the various mutations that are likely to have occurred in stealth virus related proteins, could impart some degree of energy instability in the presence of light. Whatever the mechanism, it is clear that stealth virus cultures will frequently lead to the production of auto-fluorescent material.

The underlying molecular hypothesis for the origins of stealth viruses is the initial loss of specific genes that, if present, would code for the major viral components required for effective immune recognition of the viral infected cell by anti-viral cytotoxic T lymphocytes (CTL). For many viruses, relatively few components act as effective targets for cell-mediated immunity (8). For example, in the case of cytomegaloviruses, the majority of anti-viral CTL are directed against a protein termed UL83 and most of the remaining CTL are directed against either the UL55 gene product or the immediate-early antigen. The UL83 and UL55 genes were not detected in a stealth adapted virus derived from an African green monkey simian cytomegalovirus (SCMV), while the immediate-early gene showed numerous mutations (8). Stealth viruses appear to retain and/or regain their replicative and cell damaging activities by incorporating additional genes from other viruses, infected cells (9) and even from bacteria (7). This has been clearly shown for the prototype SCMV-derived stealth virus. Many of the cellular and bacterial genes identified in cultures of this particular stealth virus show evidence of having undergone significant mutations that may well influence their biochemical and biophysical properties. These mutations may contribute to their becoming auto-fluorescent.

Of relevance to cancer development, is the potential capacity of stealth viruses to incorporate growth controlling cellular genes that are typically over-expressed and/or mutated in different types of cancer cells. These genes are referred to as oncogenes. The presence of stealth virus assimilated oncogenes can account for the high rate of recovery of stealth viruses from patients with various types of cancer. In the SCMV-derived stealth virus there are three copies of a melanoma growth stimulatory activity (MGSA) gene that have been incorporated adjacent to viral genes (6). Although this patient has yet to develop cancer, it is known that this particular oncogene is over-expressed in various types of cancers including melanomas (Haghnegahdar H et al. J Leukoc Biol 2000;67:53-62), gliomas (Robinson S. Neurosurgery. 2001;48:864-73), prostate cancer (Moore B B. Am J Pathol. 1999; 154: 1503-12), colon cancers (Yang S K. Gastroenterology. October 1997;113(4):1214-23. ), thyroid cancer (Aust G. J Endocrinol 2001; 170: 513-20), and others. The SCMV-derived stealth-adapted virus also has multiple copies of a type of chemokine receptor gene that has also been implicated in cancer development (Pati S. J Virol. 2001; 75: 8660-73).

Because they can capture, amplify and mutate genes of viral, cellular and bacterial origins, stealth viruses are easily misidentified as various types of conventional viral and bacterial pathogens. Positive stealth viral cultures are commonly found in patients diagnosed by their own clinicians as having chronic Lyme disease, chronic mycoplasma infection, and human herpesvirus-6 infections (1). These clinical diagnoses are typically based on the results of assay systems that do not exclude false positive results resulting from the presence of a stealth virus infection (1). This specification is intended to include all patients in whom it could be shown that they would give a positive stealth virus culture that would show the presence of auto-fluorescent material, and in which destruction of some, or all of the cultured infected cells, could be achieved by exposure of the cultured cells to light at wavelengths capable of inducing the auto-fluorescence.

A characteristic finding with all stealth virus cultures is that the CPE tends not to be progressive, especially in infrequently fed cultures. Relatively normal appearing cells can emerge from the periphery of cell clusters. Swollen vacuolated single cells can also revert to more normal appearing elongated fibroblasts. Providing the cultures remain unfed for several days, individual cells and cell clusters also tend to partially localize their pigmented intracellular deposits, along with an overall reduction in the extent of intracellular pigmentation. Marked cellular damage and additional pigmented accumulations can be readily re-induced in the cultures by simply replacing the tissue culture medium with fresh medium. This effect begins within minutes and progresses over 1-2 hours. It appears to be due to the removal of CPE sparing inhibitors that accumulate in the medium being used to culture the infected cells. Separate research work from this patent application is focused on using this activation/repair system to characterize the nature of this inhibitory material.

In the present patent application, I have used this activation/repair system to determine the toxicity of light for stealth virus infected cells. Specifically, I have tested the effect of exposing partially recovered cultures of stealth viral infected cells to light provided by either an Olympus MT-2 inverted microscope, or a Zeiss 310 laser-lighted confocal fluorescent microscope. Using the inverted microscope, the stealth virus infected MRC-5 cells were maintained in a culture tube in X Vivo 15 medium lacking phenol red. The culture was illuminated with a 50 watts microscope lamp at maximum output. No perceptible heat was felt on the upper surface of the tube from the light shining directly onto the tube. The illuminated cells were viewed through the microscope's objective for varying times from 1-6 hours. The cells directly in the light path would typically show significant retraction, rounding and even detachment from the glass. In contrast, I could not see any damage occurring to normal fibroblasts over a similar, or even longer periods of light exposure (up to 15 hours).

In other experiments, I could observe the loss of viability of a group of stealth virus infected cells exposed to white light passed through a pink filter, or exposed for to laser induced green light from the confocal microscope's helium/neon laser (543 nm wavelength). It is known that red/green light has a deeper penetrating capacity through normal tissue than does blue light. While using the helium/neon laser I could also document that the viable infected cells continued to emit significant red auto-fluorescence throughout the 10-minutes exposure interval. By that time, all of the exposed cells had died, as evidence by the loss of fine cellular movement when viewed under phase contrast microscopy.

A feature of certain stealth viruses is the capacity to be propagated in, and to induce morphological and biochemical changes in bacteria and even to cause bacterial death (7). I have investigated whether either bacterial cultures from stealth virus infected patients, and bacteria exposed to medium from positive stealth virus cultures, can provide a source of the auto-fluorescent material presumably coded by a patients' stealth-adapted viruses. I have commonly identified unusual bacteria in stool cultures from a number of stealth virus infected patients. I have also infected bacteria using supernatants of stealth virus positive cultures. Atypical bacterial colonies from stool samples, or from bacteria exposed to stealth virus culture supernatants, are selected on the basis of their showing one or more unusual growth characteristics. They have been further screened for the production of crystal-like and/or ribbon/thread-like deposits within the agar overlying or adjacent to the bacterial colony. Using this approach, I have observed numerous complex deposits forming in cultures of presumptively stealth virus infected bacteria. Many of the deposits are clearly pigmented, especially when the bacteria are grown on blood agar plates. The varying sized and appearing crystals and other structures, develop both on, and away from, the growing bacterial colonies. They become particularly prominent as the agar is allowed to dry through water evaporation. The crystals can be readily illuminated with indirect lighting, and have commonly shown a brief afterglow when the direct lighting was turned off. Crystals from several of such cultures were collected from the agar and examined for auto-fluorescence using the confocal microscope. Both argon (488 nm blue excitation and a green filter) and helium/neon (543 nm green excitation with a red filter) illumination confirmed that these bacterial-derived products were auto-fluorescent. The intensity of the emitted light varied between different structures, with some showing particularly intense red photo-reactivity. Red auto-fluorescence was seen with crystals collected from both blood agar plates and tryptic soy agar plates.

A patient (CM) who had chosen not to have breast surgery has lived with a histological confirmed infiltrating breast cancer of her right breast for over 9 years. She has taken several herbal medicines with substantial benefits in controlling, but not eradicating, her tumor. The patient has been on disability because of her breast cancer and, even more so, because of a concurrent chronic fatigue-like illness. Her son also has chronic fatigue syndrome. The patient tested strongly positive in a stealth virus culture and many of the cells showed pigmentory changes. Virus from her culture was also able to kill portions of a bacterial culture. In September 2001, the patient volunteered to receive light exposure to her breast cancer. The light was provided by a hand held flashlight with a red filter. The red filter was used because it is known that red light has a greater penetration through tissues than does blue, yellow or green light. At the time of the trial, her 4 cm cancer was causing mild discomfort and was sensitive to touch. The light was applied to the skin over the region of her breast cancer, by the patient for 30 minutes. Within 5 minutes, she reported that the sense of local discomfort became more noticeable. This soon began to resolve and was gone by the end of the 30-minutes exposure. Her usual level of breast discomfort stayed subdued for several days. More impressively, the patient noted that the circumference of her tumor mass was definitely softer, lacking its usual firmness, and somewhat reduced in size. The softening effect also lasted 2-3 days before returning to its usual state of firmness. No adverse effects were noted.

Another stealth virus positive patient with multiple lipomas (benign tumors of fat cells) as part of a disease process termed Dercum's syndrome (Reece P H J Laryngol Otol. 1999; 113: 174-6.), reported altered sensation in the lipomas within 15 minutes of exposure to bright sunlight. This effect was not due to heat since it was not observed while taking a hot bath. Both stealth virus cultures and stool bacterial cultures from this patient have produced large amounts of green and red auto-fluorescent materials.

In a preferred embodiment, one can isolate a stealth virus from blood and also from a tumor sample of a cancer patient. The virus can be grown on MRC-5 human fibroblast cells. The culture is examined for the appearance of a CPE indicative of a stealth-adapted virus. The culture is maintained and further examined for the accumulation of pigmented materials that will show auto-fluorescence when exposed to full spectrum white light, emitted for example from a mercury vapor lamp, or by sunlight. Normal uninfected cells will be run as a concurrent control to confirm their lack of auto-fluorescence. A finer definition of the exciting wavelengths for the stealth virus infected culture can be obtained using various alternative light sources that emit light at restricted wavelengths. These light sources include but are not limited to, coherent light lasers, tunable dye lasers, arc lamps using various ions and metals, plasma discharge tubes using various gases, and filament lamps. The effects of high intensity flash lighting can be compared with that of a steady lower intensity light. Detailed absorption and emission spectra can also be determined using a spectrometer, and can include Raman spectroscopic analysis. The virus can also be grown in bacteria and the bacterial cultures examined for auto-fluorescence material.

Once an appropriate light source and range of wavelengths are selected, they can be used to directly test the phototoxicity of the light on the abnormal cells in the stealth virus culture. This can be determined morphologically under phase contrast microscopy. Based on the findings, one can arrange for an effective light source to be applied to the patient to illuminate in vivo cancer cells. A wide variety of methods exist to administer the light. It can be targeted directly to a known site of the cancer, either from outside of the body, or from within the body, for example using endoscopy or peritonoscopy. The intensity of light exposure can be based on the in vitro culture findings and the desired depth of light penetration. Exposure of blood flowing through a single limb, using an external light source, or an intravascular light source, will eventually involve much of the entire blood volume because of the blood circulation. Alternatively, blood can be passed extra-coporally through a light-emitting device and returned to the body.

The effect on light therapy on tumor size, consistency and other changes can be determined and used to schedule the length, intensity and frequencies of subsequent light therapy sessions. Progress can also be ascertained by conducting fine needle aspirates of the tumor before and after light therapy sessions and ascertaining evidence for tumor cell death. Beneficial effects on a local tumor will encourage the use of similar light therapy to empirically destroy potential metastatic cancer deposits throughout the body.

Stealth virus can reside in bowel bacteria. Light mediated removal of infected bacteria from the body should prove useful at reducing virus burden and potentially toxic bacterial products. Blood products and tissue grafts could also be treated with light therapy as a means of destroying any stealth virus infected cells in which there is photosensitive material.

In administrating systemic light therapy to certain stealth virus infected patients, an effort may need to be made to shield the brain from the therapeutic light source. This issue is under consideration since intense intracranial light exposure might have a potentiating effect on any ongoing stealth virus induced damage to brain cells. For this reason, I am currently not suggesting the use of light for stealth virus infected patients with advanced degenerative neurological illnesses.

The prominent production of cholesterol-like crystals in certain stealth virus cultures is consistent with a potential role of stealth viruses in the formation of atheromatous plaques, as for example occurs in coronary artery disease (Ismail A. et al. Heart Dis 1999; 1: 233-40). A theoretical adverse effect of light-mediated systemic stealth virus therapy, is the possible induced alterations in a coronary artery lesion that involves the active presence of a stealth virus infection. Again, it is possible to shield selected regions of the body from any therapeutic sources of light. At the present time, avoidance of light exposure to the heart in a stealth virus infected patient suspected of having coronary artery disease, may, therefore, be advisable. Resolutions of this and other safety issues will come from experience gained in the use of light therapy for stealth virus infected humans and animals.

While stealth virus associated cancer is the most obvious application of detecting auto-fluorescent material in a stealth virus infected cell, the methods described can be applied to treating humans and animals manifesting other types of clinical illness resulting from a stealth virus infection. It is known that stealth viruses can produce widespread multi-system damage affecting various organs and body systems. Stealth virus infected patients may have endocrine, immunological, metabolic, auto-immune and other disorders (1,2). Although the pathological basis for how stealth viruses are inducing these diseases is not fully understood, it would reasonable that at least under some circumstances additional damage to infected cells might actually improve the clinical condition by removing possible sources of toxic and/or antigenic materials. The judicious use of light therapy in such patients could help resolve this issue. Certainly, stealth infected cell destructive light therapy could delay the further progression of disease due to continued viral replication in infected cells.

This approach described in this patent application differs from the conventional method of photodynamic cancer therapy, which relies on trying to render cancer cells auto-fluorescent by administrating a photosensitizing compound. Typically either 5-aminolevulinic acid that acts as a precursor for protoporphyrin IX synthesis, or porfimer sodium, which is an analogue of protoporphyrin IX, are used. The wavelength for excitation is that which is known to provide maximum excitation of protoporphyn IX In the case of this application, no photosensitizing agent needs to be administered. Instead, one is taking advantage of the finding that stealth viruses can induce the formation of various photosensitizing materials within infected cells. The types of auto-fluorescent materials being produced can vary among stealth virus isolates, and therefore, between different patients. Based on actual cultures, the absorption and emission spectra, can be determined for each isolate and used for the selection of an appropriate wavelength for light therapy. Fortunately, the auto-fluorescent materials identified to date in stealth viral cultures appear to have a rather wide range of excitatory frequencies, that may allow for flexibility of the light delivery methods and sources.

The focus of this application has been the excitatory energy of light on the abnormal cellular products accumulating in stealth virus infected cells. Other forms of energy can have modulating effects on the excitation of auto-fluorescent molecules. For example, the fluorescence of some photosensitizers can be enhanced with ultrasound (Miyoshi N. Ultrason Sonochem. 2001; 8: 367-71). Molecularly excitability, with transitioning between various energy states is one of the fundamental properties of auto-fluorescent molecules. Such molecules can often be shown to have a more general function of being able to absorb various different forms of energy and convert the energy into other forms and/or into chemical processes (Vsevolodov N. Biomolecular Electronics: An Introduction via Photosensitive Proteins. Birkhauser, Boston 1998). The production of free radicals and/or the transporting of various toxic ions, are likely to accompany the energy conversion reactions of these molecules induced by a variety of activating energy sources. I can confidently predict, therefore, that some of the a typical auto-fluorescent and other structures identified in stealth virus infected cultures and bacteria, may be effectively targeted by a variety of forms of energy exposures, beyond simply that of light. These experiments will follow the same outline as that described in this application for light. That is, cultured stealth virus infected cells can be subjected to different forms of energy emissions and examined for loss of viability (and possibly the re-emission of one or other form of energy). Compounds that show auto-fluorescence when exposed to light can be tested for light emission when exposed to other forms of energy. This can be achieved, for example, by placing the compounds on photographic film in a light-proof container. The container is placed in an electric, magnetic or radio-frequency energy field, or exposed to ultrasounds. By using varying frequencies, one can see if any of these energy sources were able to lead to light activation. Although these experiments have yet to be performed, it is reasonable to extrapolate the utility of the present invention using light, to the utility of using other forms of energy radiations to treat stealth virus infections. These could include the use of radio-frequency, ultrasound, and magnetic fields, administered alone or in combination; and with or without the addition of light. The basic principle is that stealth virus infections can lead to the accumulation within the infected cell of abnormal molecules, and that these molecules can potentially be selectively targeted with various excitatory energies that can cause destruction of the infected cells.

An interesting aside from the present application is the potential of using purified red auto-fluorescent materials produced by stealth virus infected cells and bacteria as a photosensitizer in studies outside of stealth virus therapy. Protoporphyn IX auto-fluorescence has not proven itself to be particularly useful in clinical studies and there is a need for alternative auto-fluorescent products, especially those that can give red auto-fluorescence. Molecules that auto-fluoresce are also finding basic research applications.

### EXAMPLES AND ILLUSTRATIONS

The findings described in this application have been derived mainly from numerous detailed examinations performed on i) stealth virus cultures, ii) bacterial cultures infected with stealth viruses, and iii) tissues from stealth virus infected humans and animals. The culturing of stealth viruses is not yet a generally accepted method of research and/or clinical testing. For this reason, I have included a larger number of photographs than might ordinarily be required in a patent application. The photographs are included to especially show the type of CPE that is commonly seen in stealth virus cultures, and also to document the occurrence of a typical auto-fluorescent cells and extracellular structures. They also provide evidence for the presence of a typical structures in the tissues of stealth virus infected patients. Photographs of the cell damaging effects of light on stealth virus cultures are also included.

Topic A: Cytopathic Effect (CPE) of Stealth Viruses in Tissue Culture:

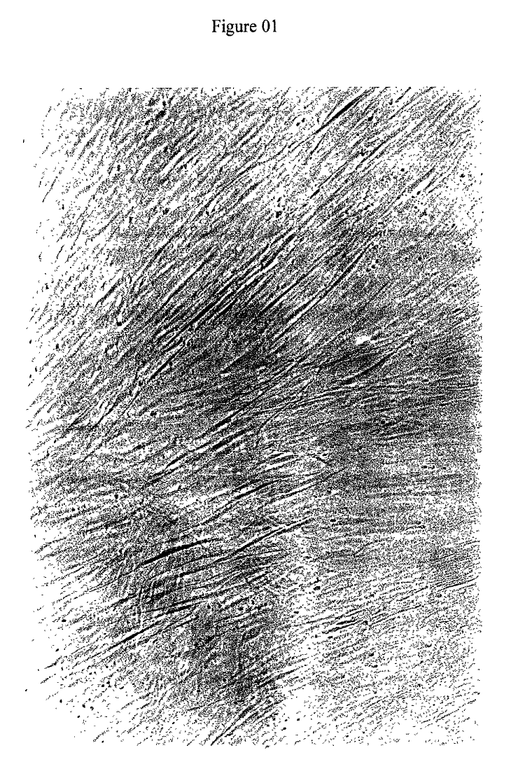

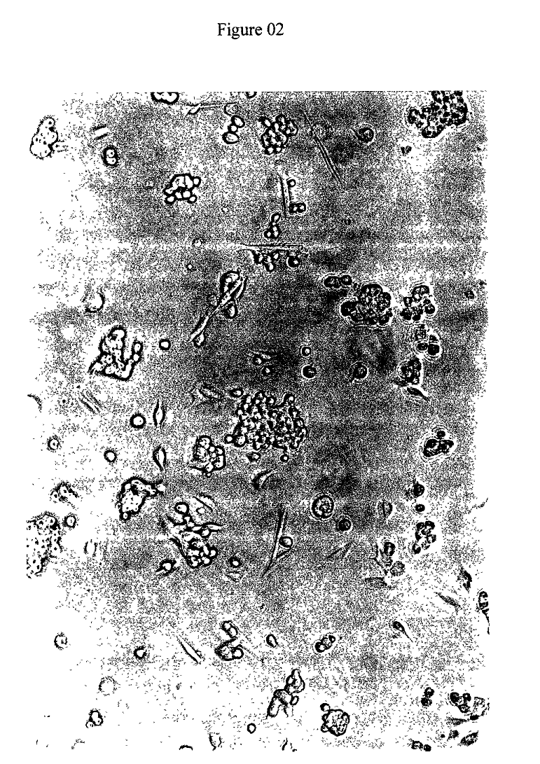

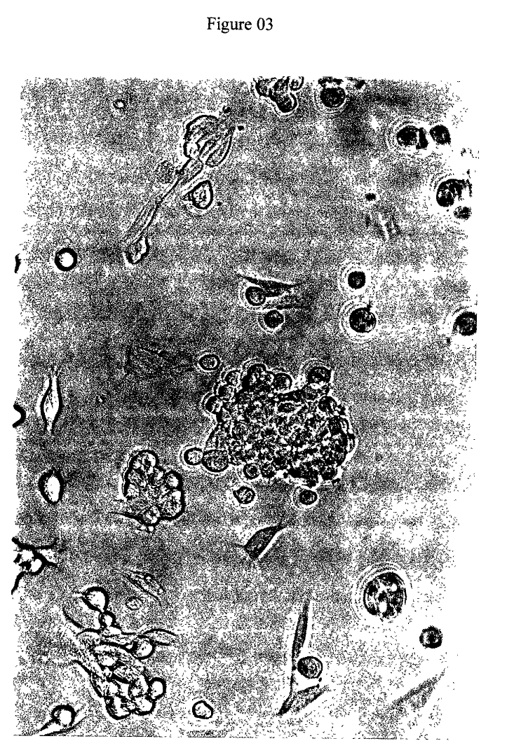

FIG. 1 shows the low power (10× objective) microscopic appearance of normal MRC-5 fibroblasts. The cultures consist of closely packed, interdigitating, spindle shaped, non-pigmented cells, that form a flat, rather bland appearing, cell monolayer. FIGS. 2 and 3 show the low and higher power (20× objective) of a clearly positive culture. The changes shown occurred within a week of adding a frozen-thawed extract of mononuclear cells obtained from a stealth virus positive patient. In the first, one to several days, many of the cells began to change from a spindle to a more rounded shape. There was an increase in the amount of cytoplasm, which acquired a foamy, somewhat vacuolated appearance. Many of the cells disappeared from the culture with the remaining cells tending to adhere to, and even to fuse with each other. As the strongly positive cultures developed, more and more of the surviving cells collected into various sized clusters with relatively few intervening surviving cells.

Topic B: Presence of Extraneous Products in Stealth Virus Cultures:

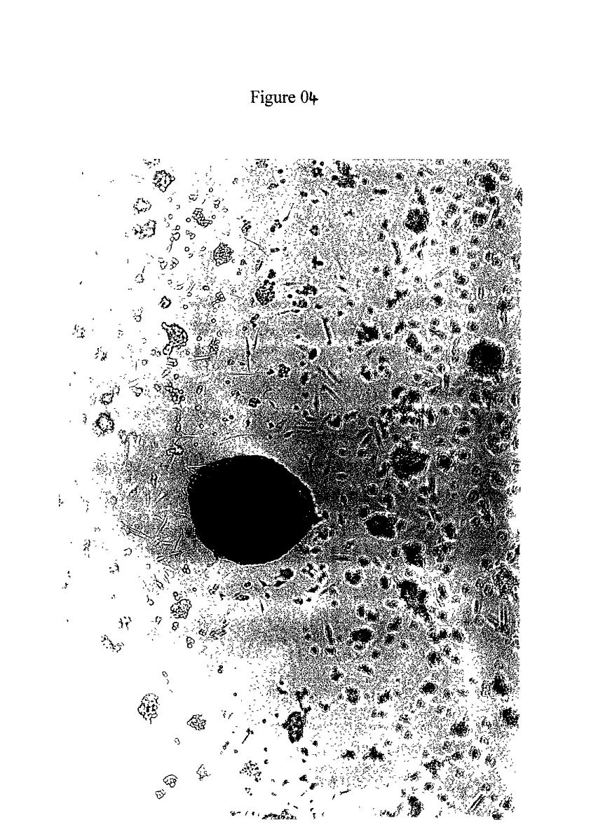

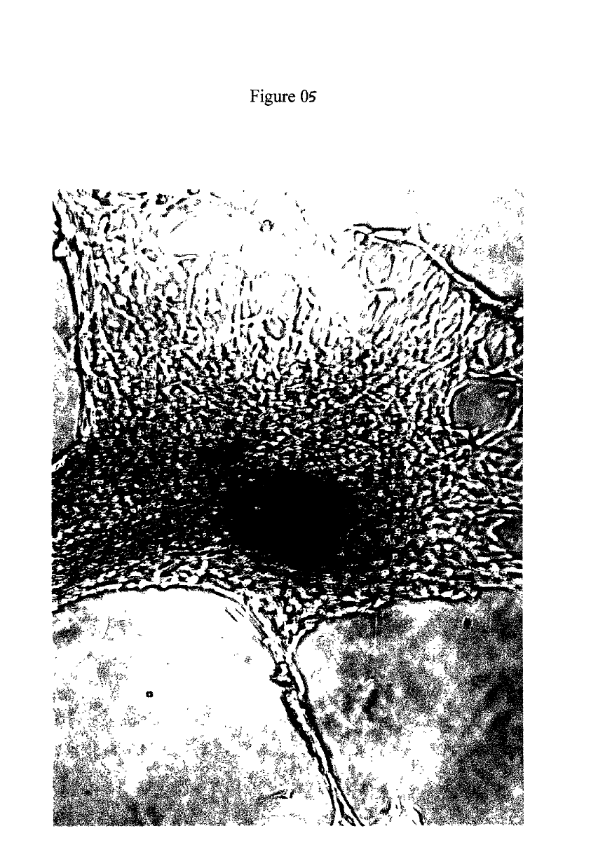

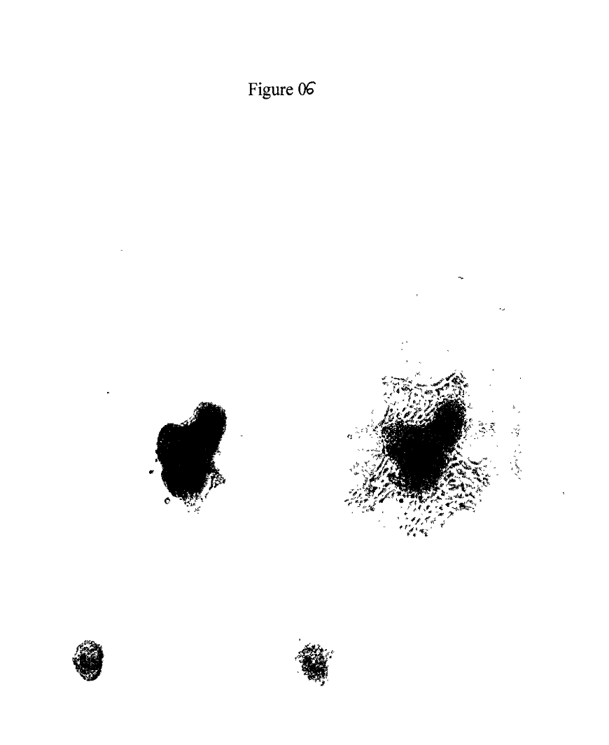

Over 1-2 weeks, many of the cell clusters will acquire a degree of brownish pigmentation. Especially in some of the cultures, the extent of pigmentation can be quite striking, as seen for example in FIG. 4. The pigment can vary from a light yellow-brown color to a much darker black. It can be finely or coarsely granular, or exist as a diffuse color change. Over several days to a week or more, the pigment will often become localized within the cluster (as shown in FIG. 5). This occurs, especially when the cultures are left without replacement of the tissue culture medium Moreover, as shown in FIG. 6, relatively normal appearing cells can emerge from many of the cell clusters, providing the medium is left unchanged.

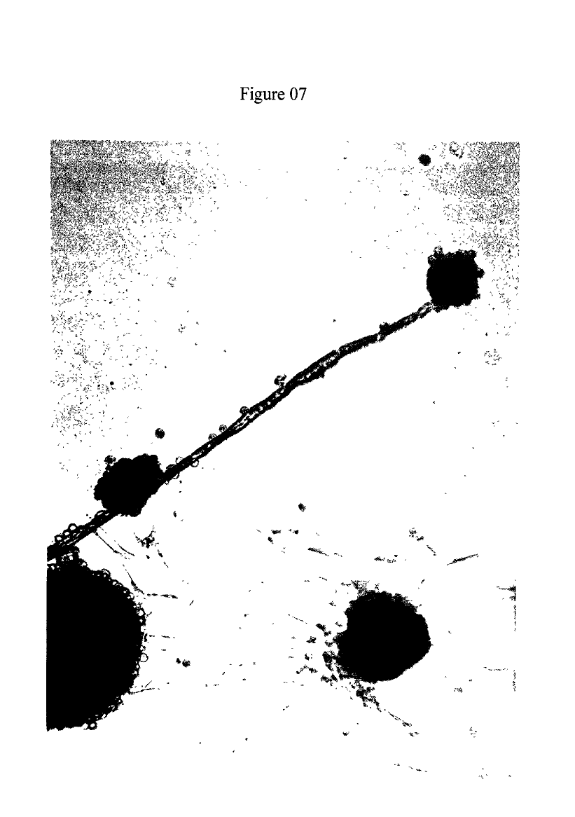

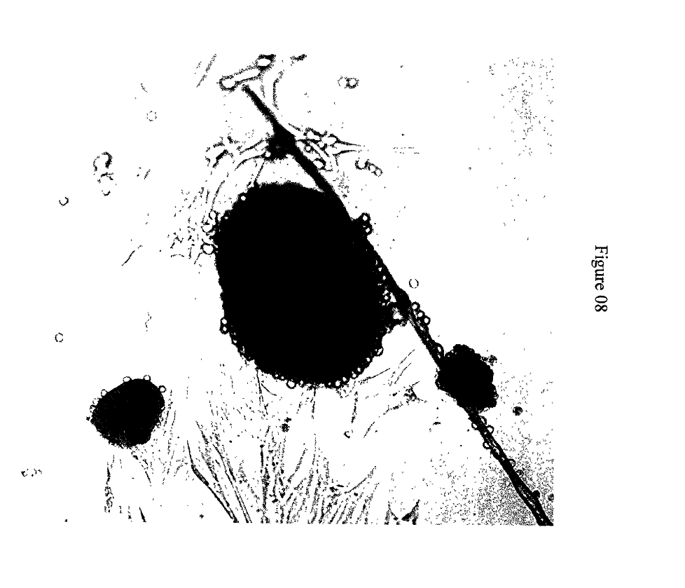

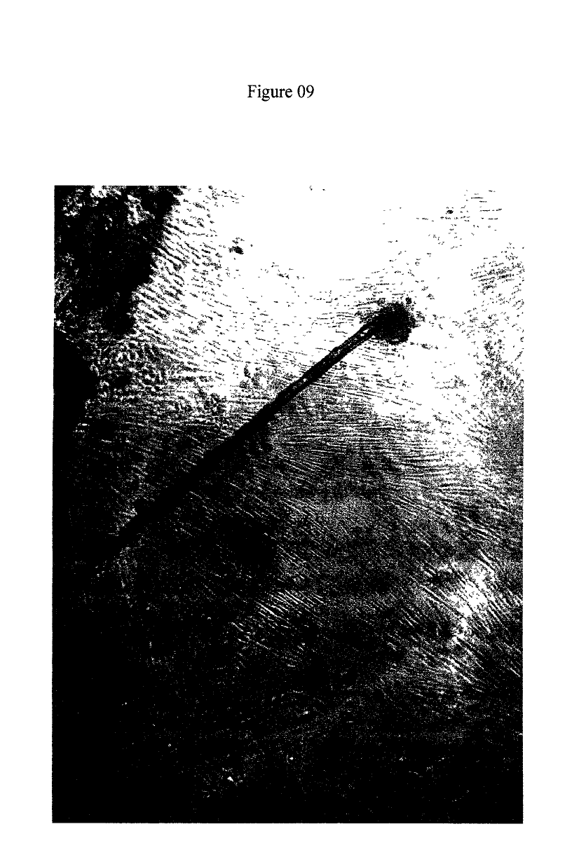

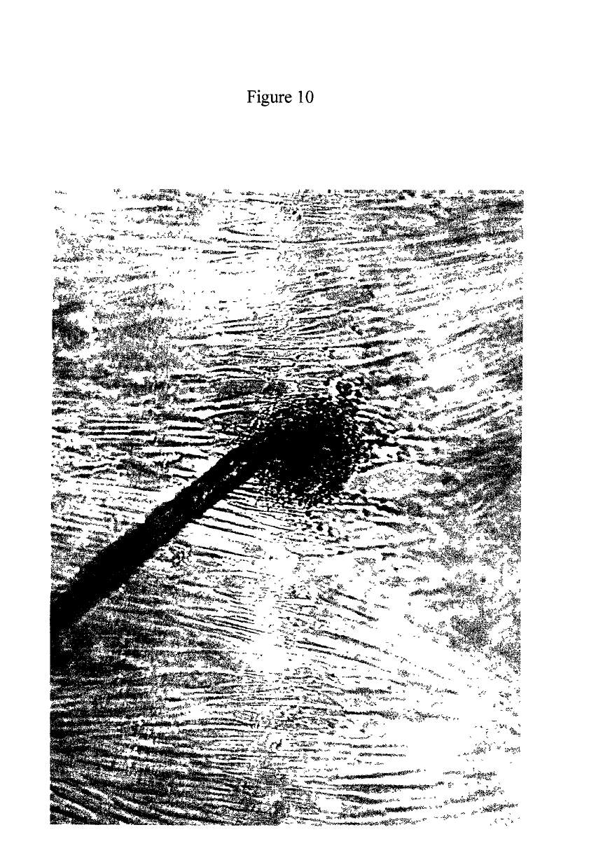

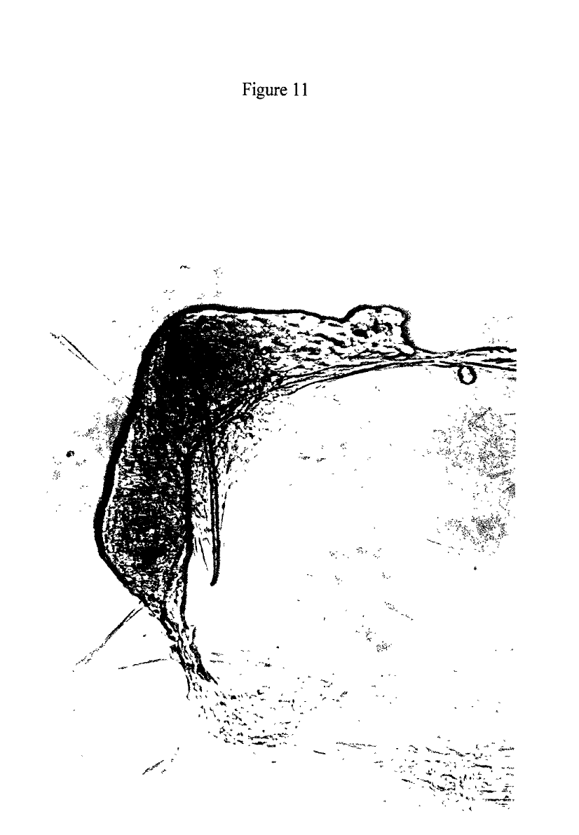

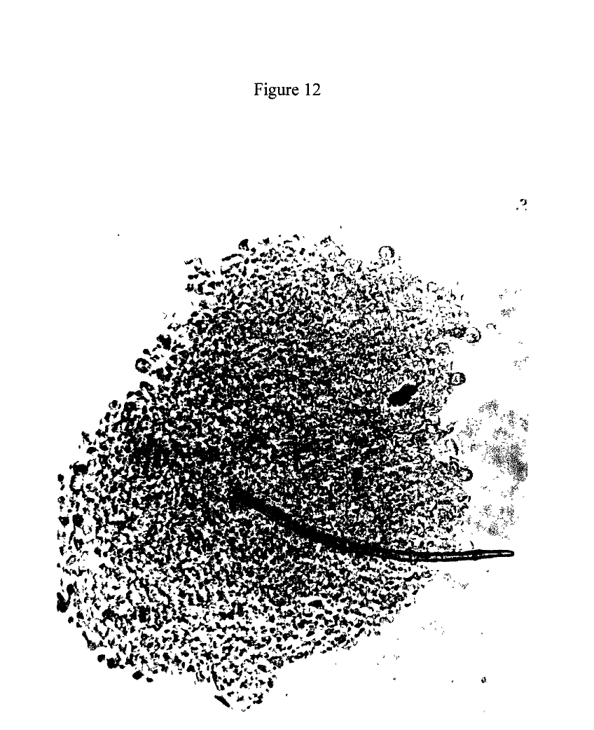

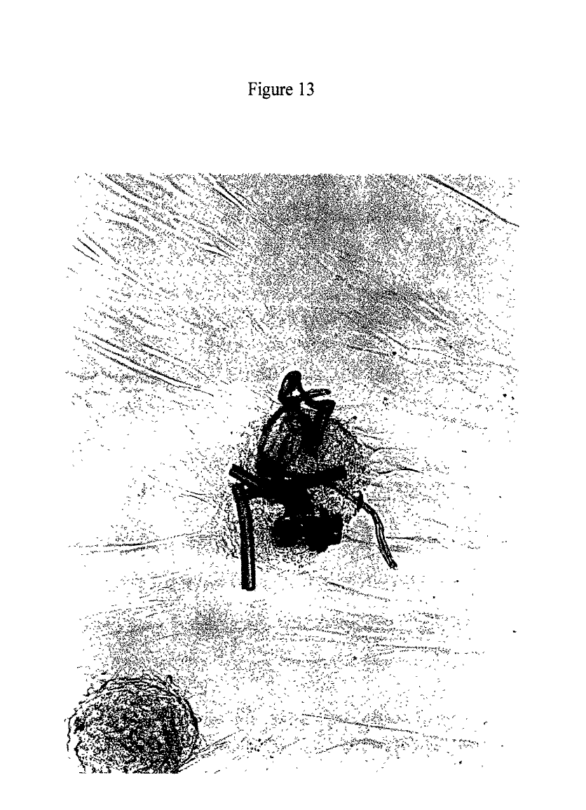

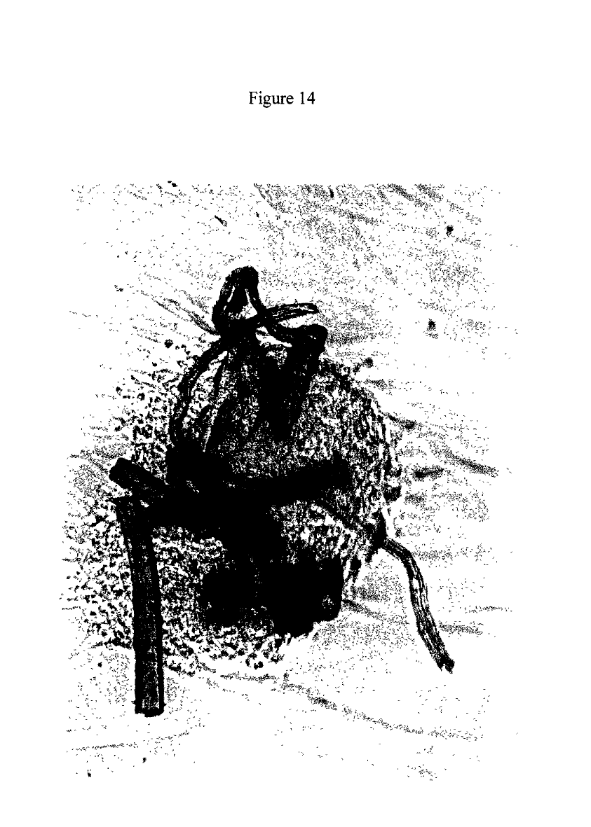

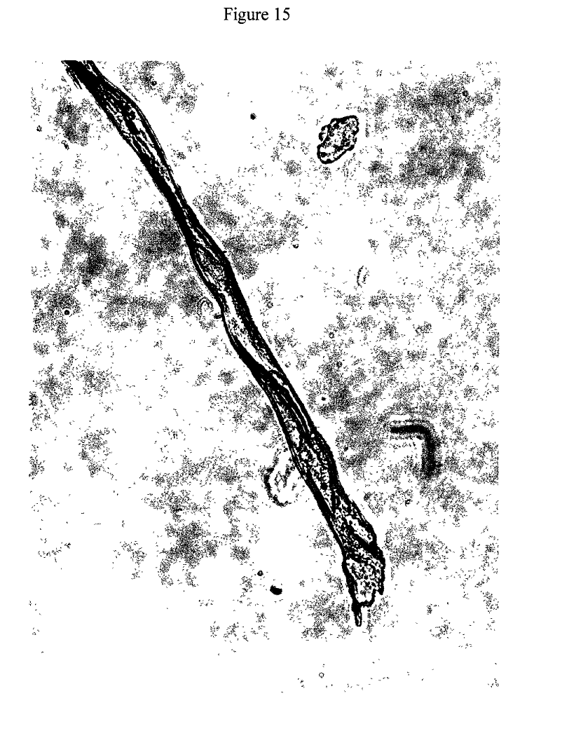

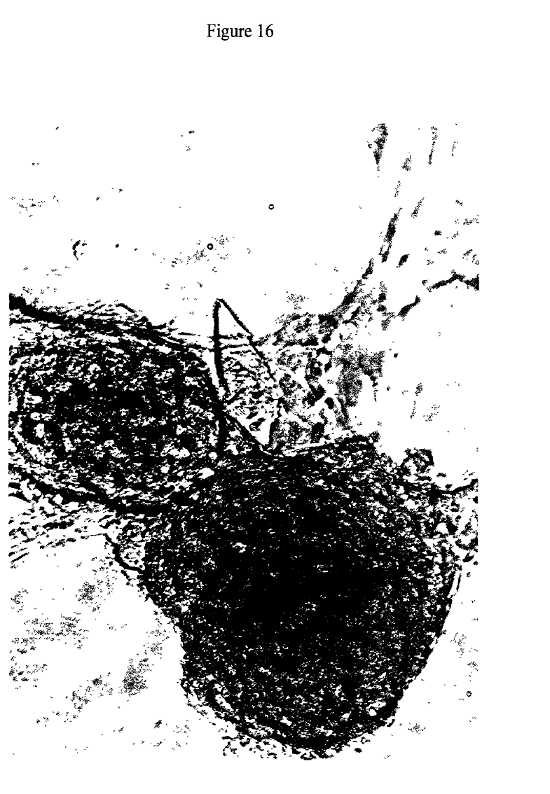

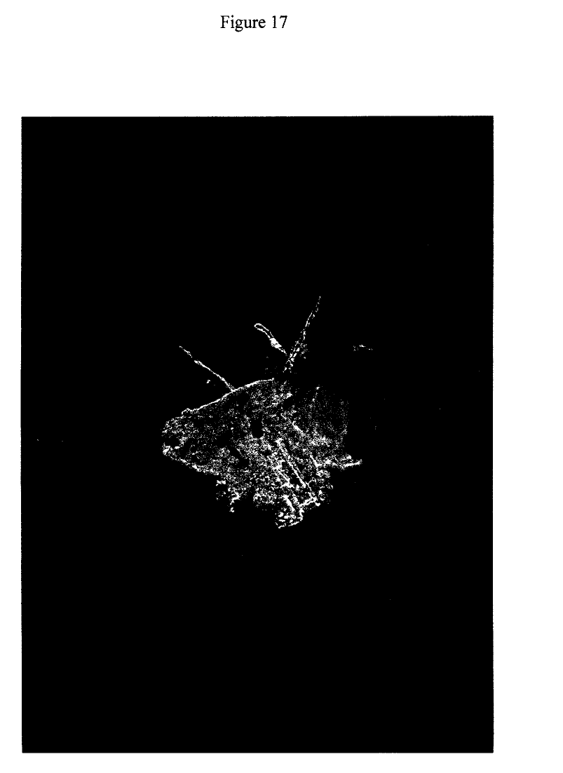

A striking feature in many of the more strongly positive cultures is the formation of extra-cellular structures that appear to arise in cells, but clearly also grow outside of the cells. These structures can take the form of both long thin threads and more tubular and/or ribbon appearing protrusions. An example of a growing long thread-like structure emerging from the relatively small cell cluster is shown in FIGS. 7 and 8. Long thread-like structures can continue to grow in spite of considerable repair occurring elsewhere in the culture. This is seen for example in FIG. 9 (low power) and FIG. 10 (high power). Other examples from different cultures are provided in FIGS. 11 and 12. The structures are not a uniform features throughout a given culture, but are typically seen arising from only an occasional cell cluster. This can be seen, for example, in FIG. 13 (low power) that shows one cell cluster with multiple small ribbon-like structures, along with patches of residual pigment. A nearby cell cluster has minimal pigmentation and no ribbon-like structures. A higher magnification of the complex ribbon-like structures is shown in FIG. 14. The various a typical structures can continue to grow even after becoming detached from the cell clusters and even after most of the cells have died. An example is shown in FIG. 15. Irregular shaped, translucent and colored, crystal and plate-like structures, can also occasionally be seen in stealth virus cultures. An example of a translucent crystal is provided in FIG. 16. It shows a large diamond shaped structure to the left of the junction of two cell clusters. A more complex appearing structure was photographed using dark field illumination and is shown in FIG. 17. Under white light, the lower half of this structure was pale green, while most of the remaining areas were variegated yellowish and brownish in appearance. Several small black deposits were also present. Distinct tube-like structures were seen within and also protruding from this structure.

It should be emphasized that none of these a typical structures have the appearance of any normal cell or microbe. While, they appear to be mainly protein in composition, they are generally quite resistant to 100 ug/ml protease K digestion. In this regard, these structures may be relevant to some of the protease resistant products associated with various neurodegenerative diseases (e.g., Wadsworth J D et al. Lancet 2001; 358:171-80. Dyer R B. and McMurray C T. Nat Genet 2001; 29: 270-8. Kaneko I. et al. Neuroscience 2001; 104:1003-11).

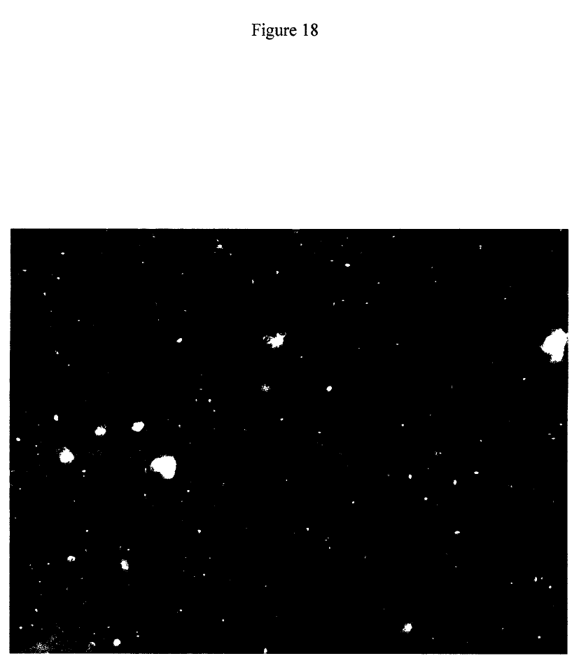

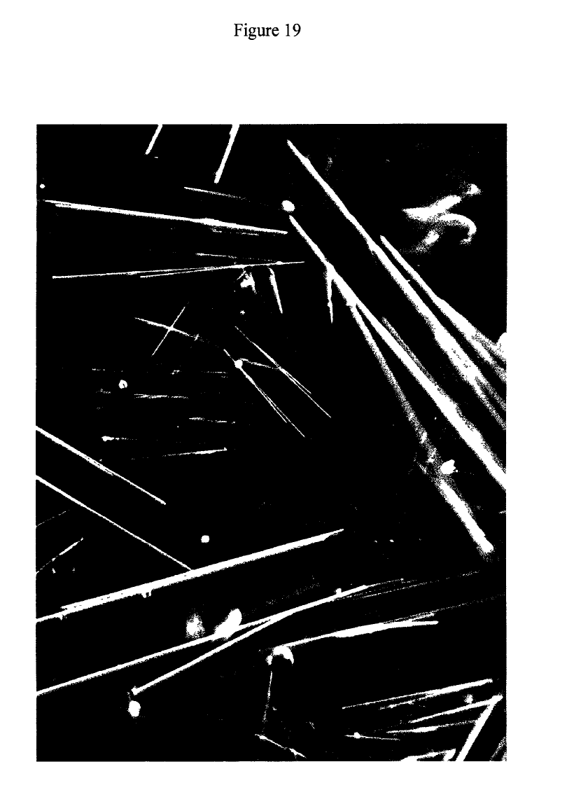

The culture medium in positive stealth virus cultures can also show widely varying amounts of fine and/or coarse particulate materials. Some of the floating material clearly corresponded to the pigmented granular materials present within many of the surviving cells. Other material had more of the appearance of lipid-like components, especially when it attaches to the glass surface of the culture tube. Furthermore, occasional needle shaped, cholesterol-like crystals could be seen. An example of the floating lipid-like particles and occasional cholesterol-like needles are shown in FIG. 18. Although, in the majority of positive cultures, the medium remains essentially clear, a large quantity of soluble extraneous material can exist in the culture medium. For example, in approximately 5 percent of positive cultures, numerous cholesterol-like needles can be precipitated from the medium by simple dilution. This is a very prominent feature of the culture from the patient with the SCMV-derived stealth virus. Whereas normal X Vivo-15 medium has no measurable cholesterol the medium from this stealth virus culture can contain greater than 10 mg/100 ml of measurable cholesterol. Moreover diluting the culture medium can lead to widespread precipitation of cholesterol-like needles, as shown in dark field illumination in FIG. 19. Crystal precipitation by dilution is consistent with the presence in the undiluted medium of lipoprotein-bound soluble cholesterol-esters. Thus for example, it is known that dilution tends to separate the lipoprotein from the cholesterol, making the latter more likely to crystallize (Kellner-Weibel G. et al. Arterioscler Thromb Vasc Biol 1999; 19: 1891-8). Various crystals, some of which are brightly colored and oddly shaped, can also be derived from various stealth virus culture media by extraction with chloroform. The lack of solubility in chloroform, and the incomplete and varying solubility in other solvents, such as acetone, propanol, ethanol, methanol, ethylene acetate, methylene chloride, and acetonitrile, are strong indications that the crystal components are not pure lipids. Rather they appear to be much more complex structures that contain one or more hydrophobic lipid components.

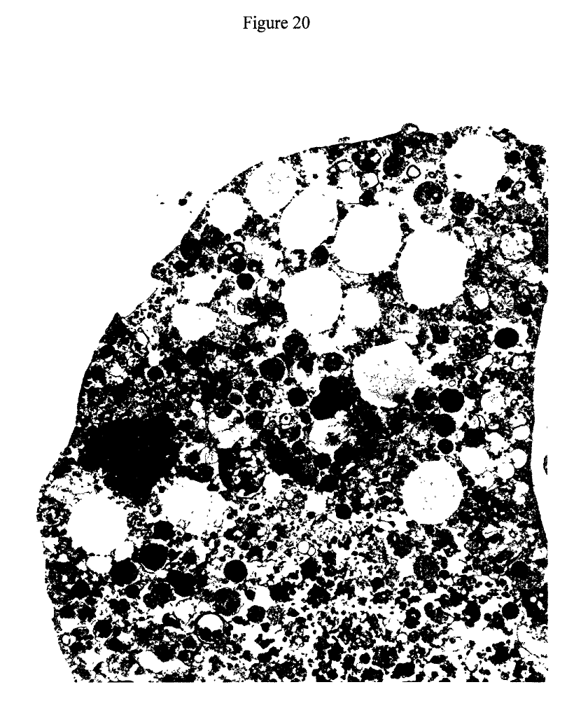

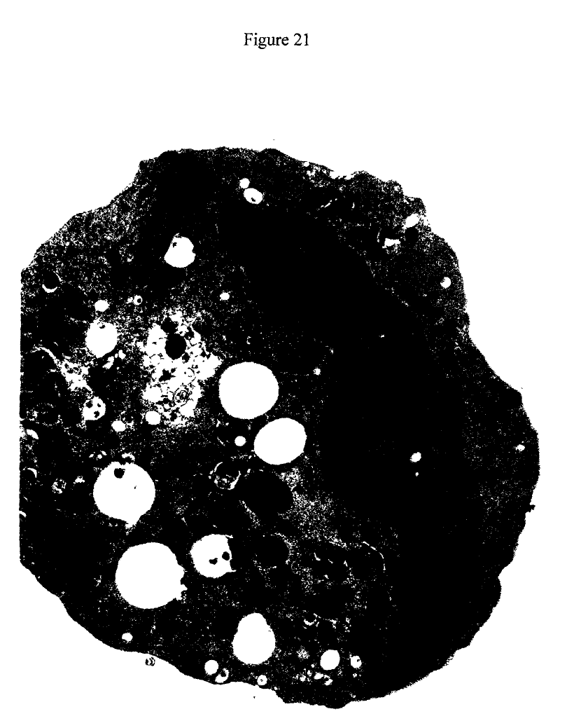

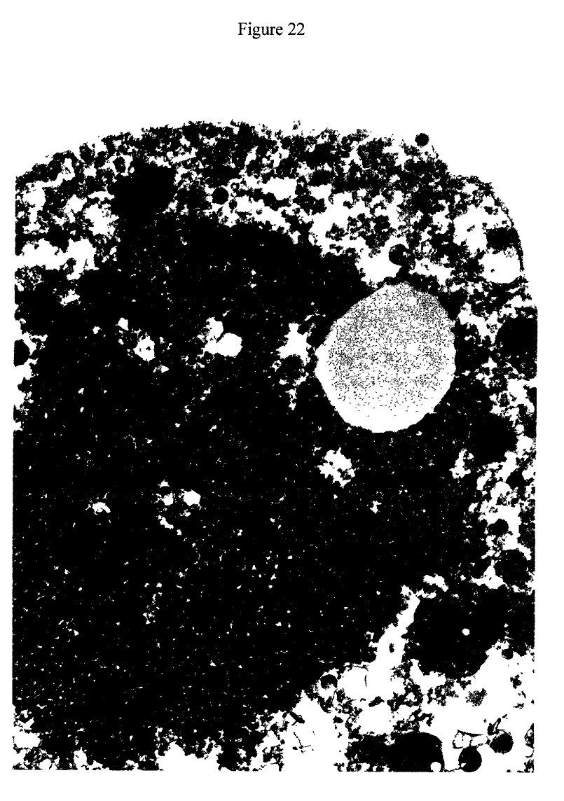

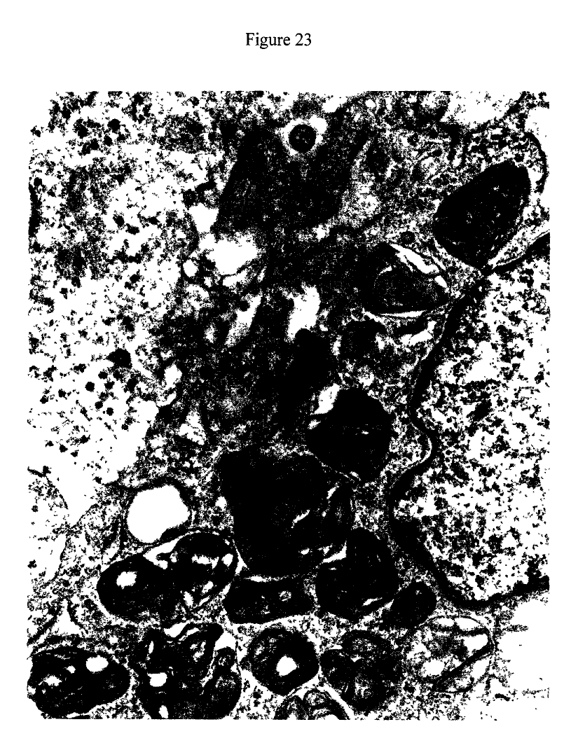

The presence of extraneous intracellular and extra-cellular structures in stealth virus cultures can also be demonstrated by electron microscopy of the cells. In addition to lipid filled vacuoles, cultured cells will typically contain a vast array of fine and coarse granular materials. This can be readily seen, for example in FIG. 20, which shows residual lipid in the larger vacuoles, and widespread deposits of varying sized particulate materials. A vacuolated cell with extensive deposits of a more uniform, coarse granular material is shown in FIG. 21. A higher power view of similar material in a different cell is shown in FIG. 22. Another feature that can be seen in some stealth virus cultures is the appearance of lipoprotein-like lamellar bodies, not unlike those in Type 2 pneumocytes of the lung (Sato S. and Kishikawa T. Med Electron Microsc 2001; 34:142-51). The particular example shown in FIG. 23 was from a culture of a child from the Mohave Valley region of Western Arizona, with a severe and eventually fatal stealth virus encephalopathy (5).

Topic C. Presence of Extraneous Material in Cultures of Stealth Virus Infected Bacteria.

I have cultured bacteria from the stools and from throat swabs of several stealth virus infected patients. Typically, I will use broadly permissive culture plates, including blood agar and tryptic soy agar (Hardy Diagnostics, CA). I also occasionally use agar plates that are more restrictive to the growth of certain types of bacteria, or that will show some specific biochemical reaction. Also, I will purposefully add a small number of either a gram positive enterococcus, or a gram negative E. coli to antibiotic free stealth virus cultures. The bacteria are harvested 1-2 days later and grown as above. The various bacterial colonies are examined microscopically using direct and indirect illumination as provided by a dissecting microscope (Leica, Model MET). Any bacterial colonies that show unusual growth behavior are selected for re-plating on agar and for characterization of their growth in thioglycolate broth. Normal bacteria will typically grow progressively in thioglycolate broth and will render the entire medium cloudy. I have repeatedly observed unusual patterns of bacterial growth using bacteria either from stealth virus infected patients, or that had been exposed to stealth virus infected cultures. In many cases the bacterial growth is confined to only a small band within the tube. It is as if bacteria moving away from this band cannot survive. Indeed, in some broth cultures all of the bacteria begin to die off leaving no visible trace of their previous existence. Similarly, when plating abnormal bacteria at a low density, it is not uncommon to see extended stretches of failed growth, well beyond what would be expected from a simple dilution effect.

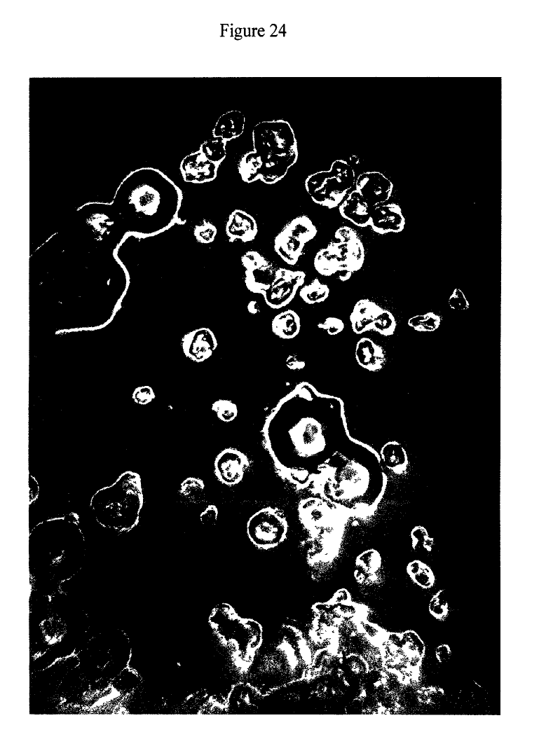

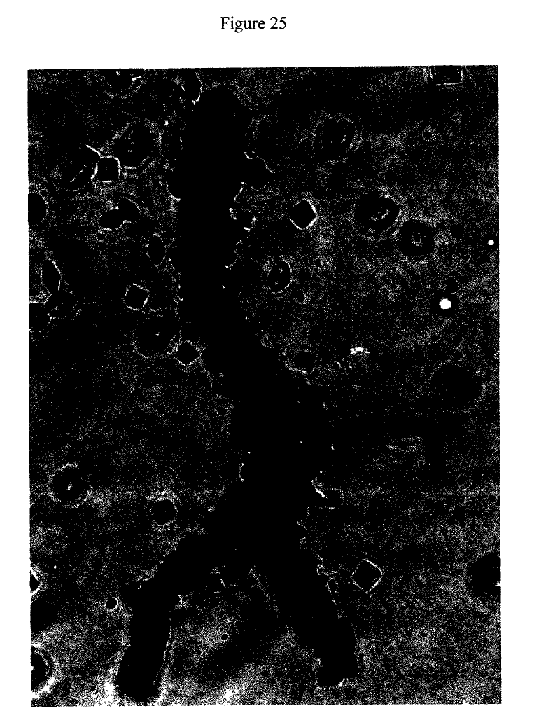

Abnormal bacteria can be tested for the presumptive presence of a stealth virus as follows: The selected bacteria are grown in antibiotic free X Vivo 15 medium. Within a day of plating the bacteria, I will prepare a freeze-thawed extract of a bacterial suspension. I will adjust the pH to 7 and clarify the material by centrifugation. The clarified material is passed through a 0.45 micron filter. The material is then tested for the ability to induce a stealth virus associated CPE in MRC-5 cells. Positive findings have been found with a number of bacterial isolates. For the purpose of this application, the major focus is on the examination of bacteria from stealth virus infected patients for the production of auto-fluorescent material. Similar examinations are made on bacteria after exposure to a positive stealth virus culture. The plated bacteria are observed for the production of crystalline and thread-like structures that will show auto-fluorescence. The bacterial colonies are examined with the help of direct and angled illumination as provided by a dissecting microscope (Leica). Shortly after the appearance of bacterial growth, I can commonly see the formation of varying sized particulate materials within the growing bacterial colonies. These structures can take the form of irregular crystals, as well as less well defined deposits. Long, thread like structures, some of which show branching can also develop. These extraneous structures will typically show some colorization that facilitates their distinction from the actual bacterial colonies. Furthermore, as time progresses, such structures will form not only in the direct vicinity of the bacterial colonies, but elsewhere throughout the agar plates. They become particularly apparent, as the plate is allowed to dry. The types of extraneous structures seen in these cultures do not develop in cultures of corresponding normal bacteria, nor can they be seen in agar plates that are simply allowed to dry. Considerable variability exists in the sizes and appearances of the types of structures seen in bacterial colonies obtained from different stealth virus infected patients. They can become quite large measuring up to 1 mm. An example of the crystals present in the agar plate used to culture bacteria derived from a stool sample of a stealth virus positive patient with amyotrophic lateral sclerosis, is shown in FIG. 24. These crystals showed mainly red colorization, but also had areas that appeared bright green, when illuminated with white light. They also demonstrated a brief afterglow upon switching off the light source. Different types of complex branching structures were seen in the culture of bacteria that had been exposed to the stealth virus culture of a breast cancer patient. An example of such a structure is shown in FIG. 25. Smaller crystals can also be seen in this photograph.

Topic D. Presence of Extraneous Materials in Tissues From Stealth Virus Infected Patients.

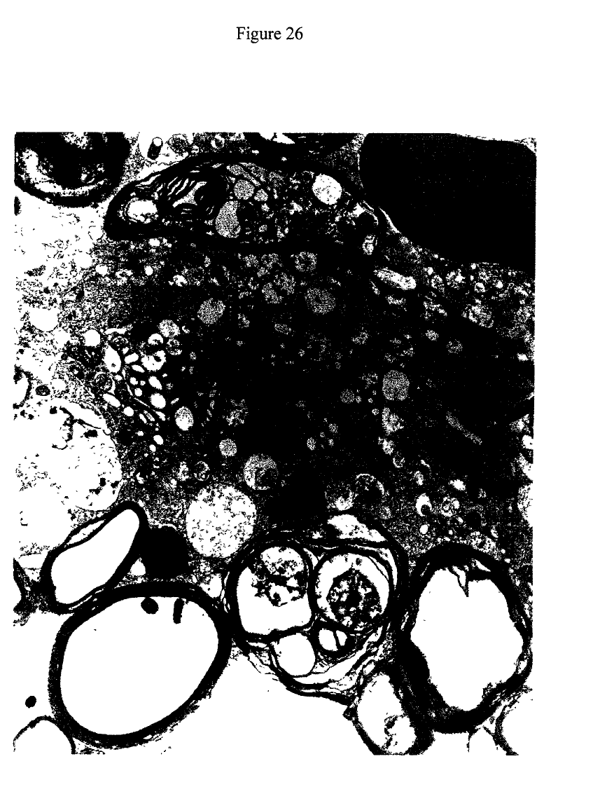

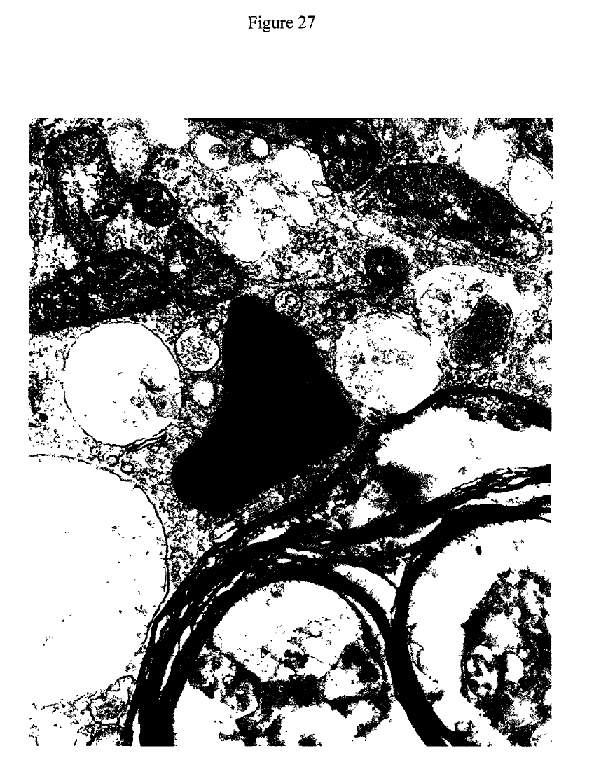

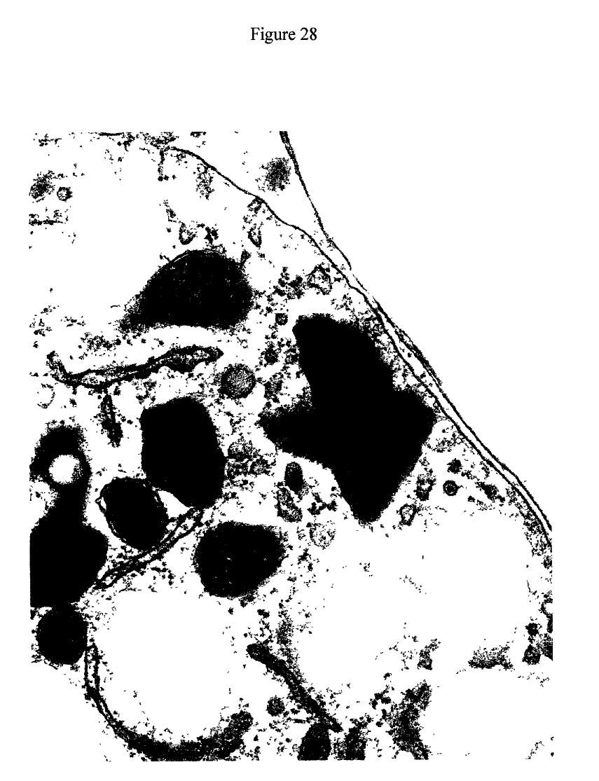

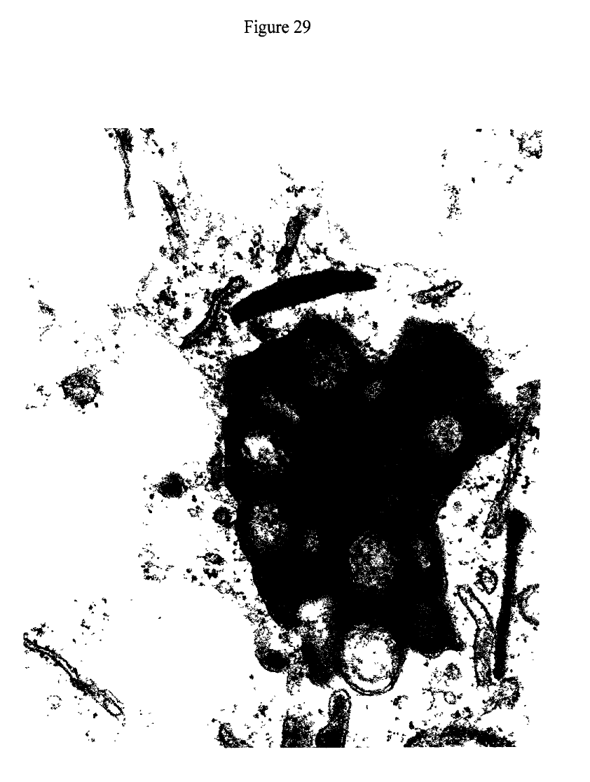

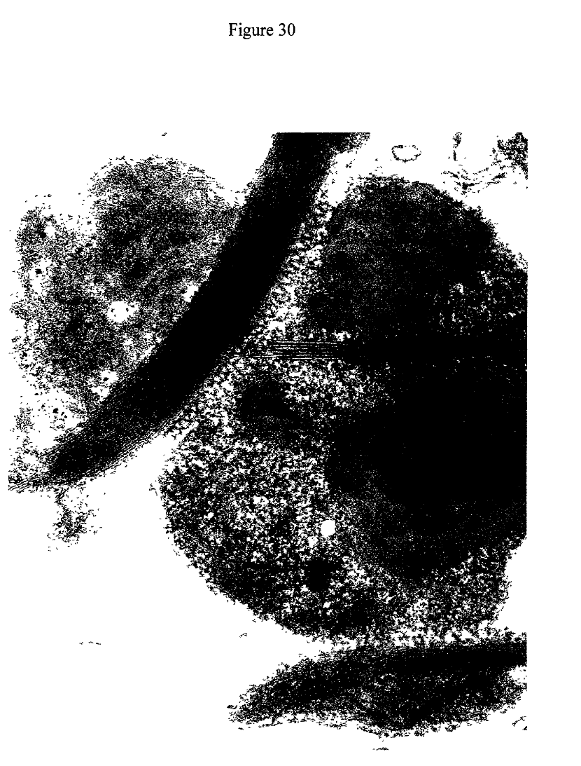

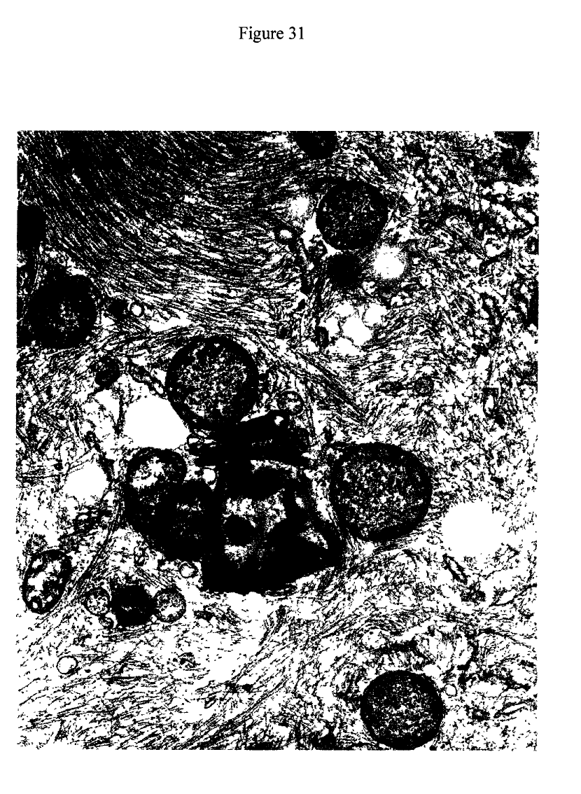

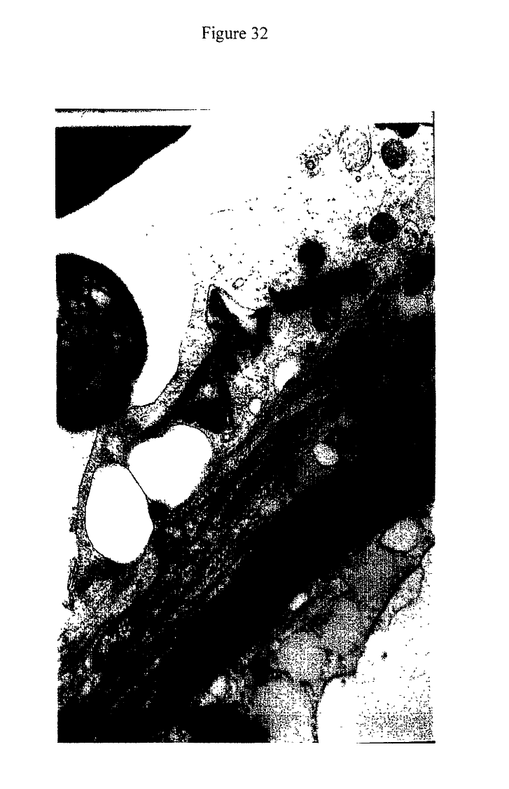

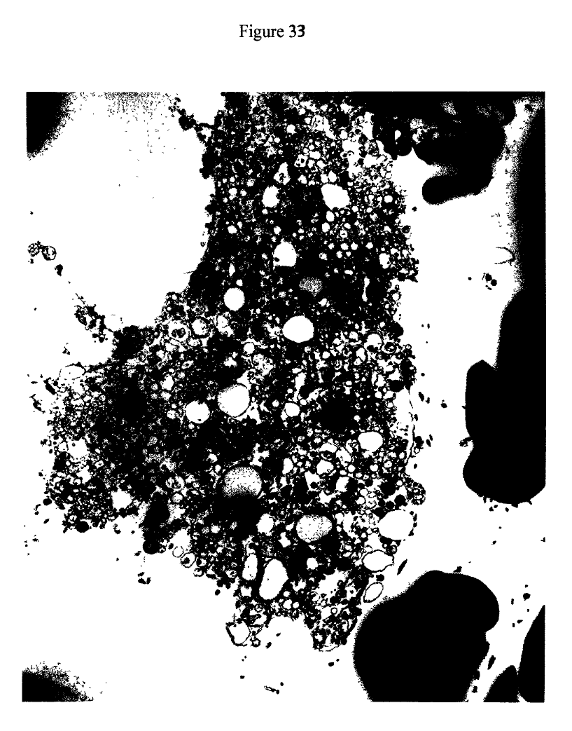

The issue of the relevance of the in vitro structures seen in cell and bacterial cultures from stealth virus infected patients to the in vivo situation is addressed in this section. Specifically, I have repeatedly identified markedly abnormal structures in tissues from stealth virus infected patients and animals. For example, as seen in brain biopsies from several other stealth virus infected patients, there were numerous intracellular inclusions within abnormal appearing cells in the brain biopsy from the stealth virus infected child from Arizona. These inclusions are best seen using periodic acid Schiff (PAS) stain, or Stains-All stain. Evidence for the structurally abnormal characteristics of various intracellular inclusions was provided by electron microscopic studies performed on the biopsy. In addition to the hallmark changes of lipid-filled, foamy vacuolated cells, mitochondria disruption and occasional cell syncytia (5), several of the cells contained peculiar structures, not unlike those seen in stealth virus cultures. In particular, there were prominent darkly staining bodies that did not correspond to any normal cell structure. A complex, unevenly stained, triangular shaped inclusion, that is adjacent to a myelin sheath, is shown at low power in FIG. 26 and at a higher power in FIG. 27. Additional examples of generally comparable structures are provided in FIG. 28 and an example of a somewhat different formation is shown in FIG. 29. A totally different type of structural abnormality is shown in FIG. 30. It comprises bands of parallel fine tubular material, imbedded in a amorphous material. Note the fine helical structure just beneath the upper band of the tubular material. Fine helical structures can also be seen adjacent to an inclusion containing irregularly shaped, densely staining material in FIG. 31. This particular cell also shows an abundance of grossly disorganized neurofilaments. A glial cell from a brain biopsy of a different stealth virus infected patient is shown in FIG. 32. Again, one can see multiple intracellular deposits of unevenly shaped, heaving staining cellular debris-like material, similar to that which can occurs in various stealth virus cultures. An in vivo example of a stealth virus infected cell from outside of the brain is shown in FIG. 33. This cell was obtained from a fine needle aspirate of the submandibular gland of a stealth virus infected patient. Varying sized granular deposits are seen within the heavily vacuolated cells. The black structures outside of the involved cell are erythrocytes.

Topic E. Auto-Fluorescence of Cells and Extra-Cellular Materials Present in Stealth Virus Cultures.

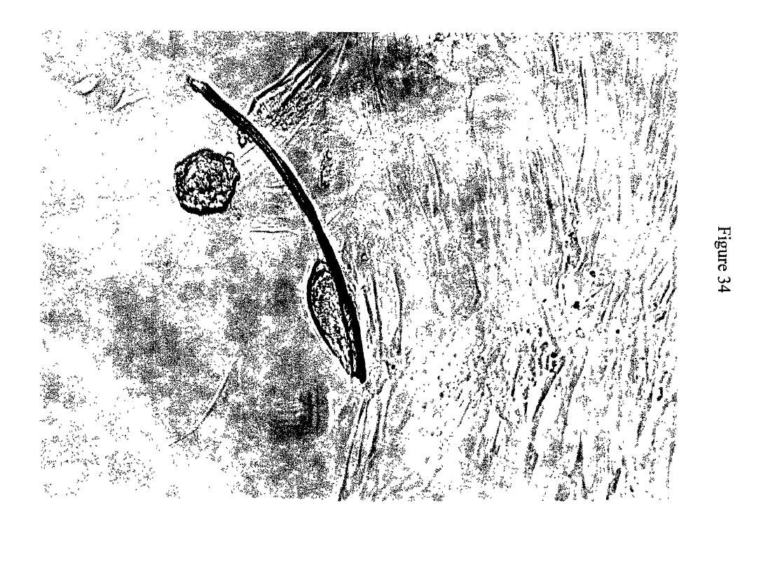

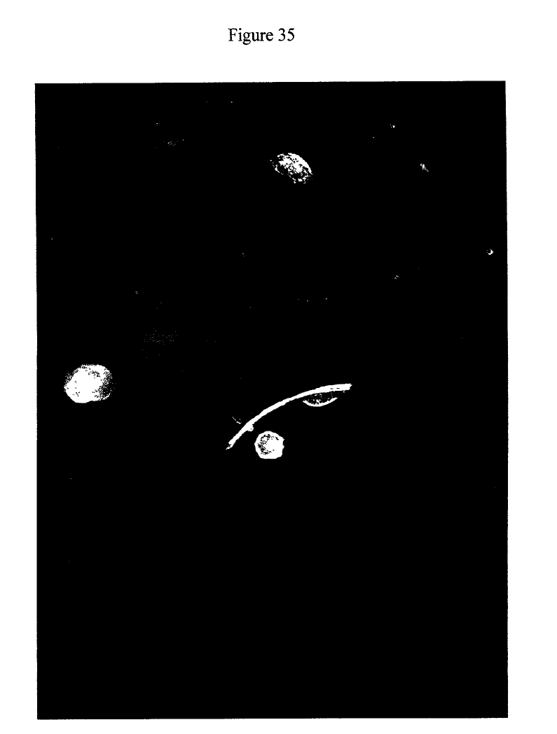

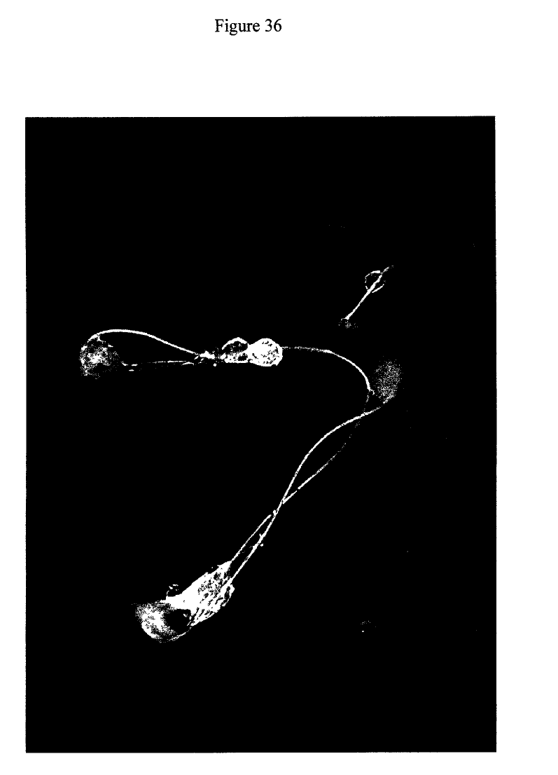

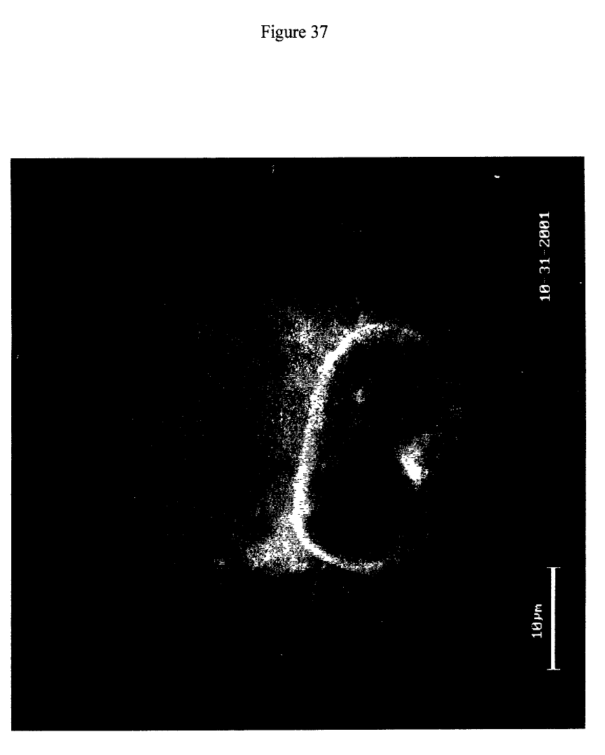

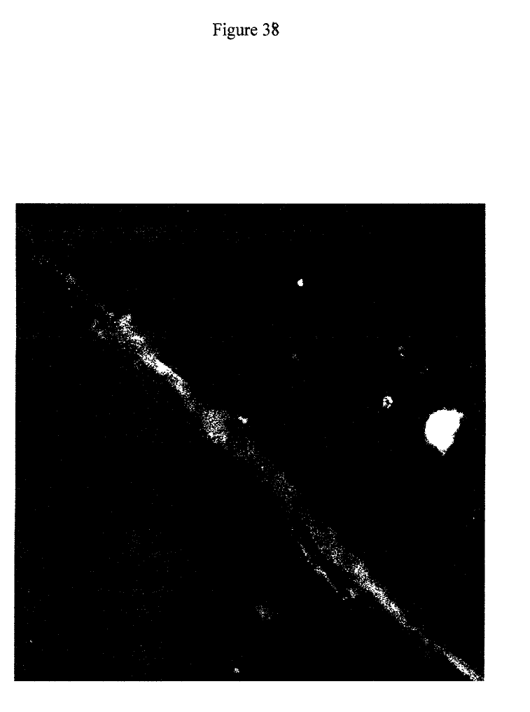

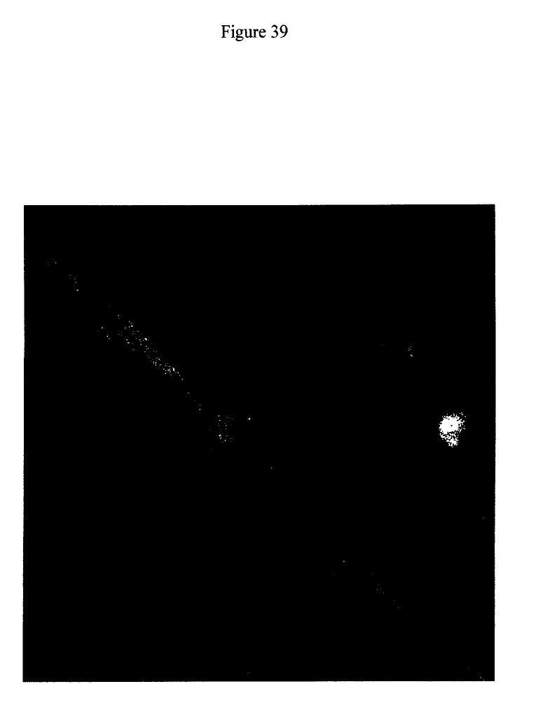

FIG. 34 shows the regular phase contrast image and an auto-fluorescence image (FIG. 35) of a stealth virus culture. Note the bright light being emitted from the cluster of cells above the tube-like structure and from the tube-like structure itself. Fluorescence is also seen along the edge of the cluster of cells from which the tube-like structure is originating, and also from particulate material within the cluster of cells at the left side of the photo. FIG. 36 shows auto-fluorescence of a long thread-like structure and of the attached cell clusters. A bi-nucleate, rounded, stealth-virus infected MRC-5 cell was studied for auto-fluorescence using a confocal microscope and either blue or green laser excitation. Considerable fluorescence was seen, especially through a red filter with green light excitation (FIG. 37). It was particularly prominent around the membrane of the fused nuclei. The fluorescence seen was generally non-uniform throughout the cells and extra-cellular structures. For example, only a patchy pattern of green auto-fluorescence was evoked using the blue (argon) laser to visualize a prominent tube-like structure seen in a stealth virus culture (FIG. 38). The structure showed somewhat more prominent, but still patchy, red auto-fluorescence, when illuminated with the helium/neon (green) laser (FIG. 39).

Topic F. Auto-Fluorescence of Extraneous Materials Produced by Bacteria Obtained from Stealth Virus Infected Patients and by Bacteria Exposed to Stealth Viruses.

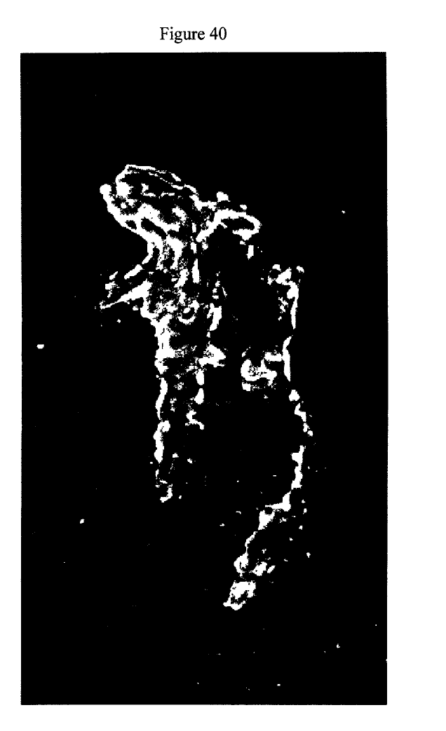

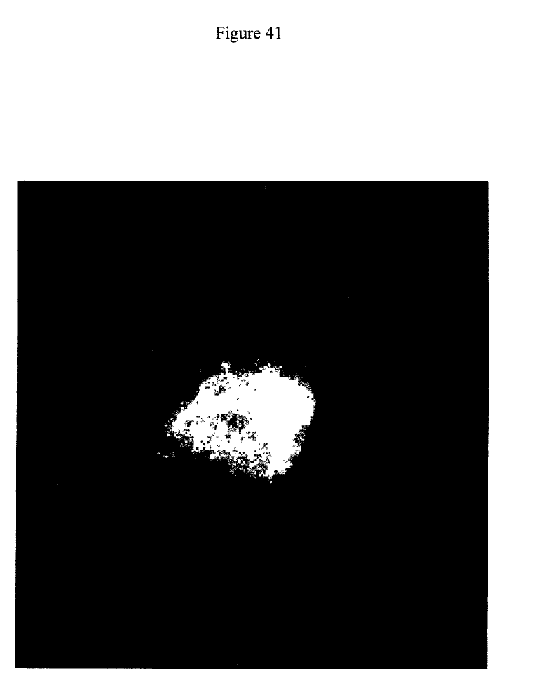

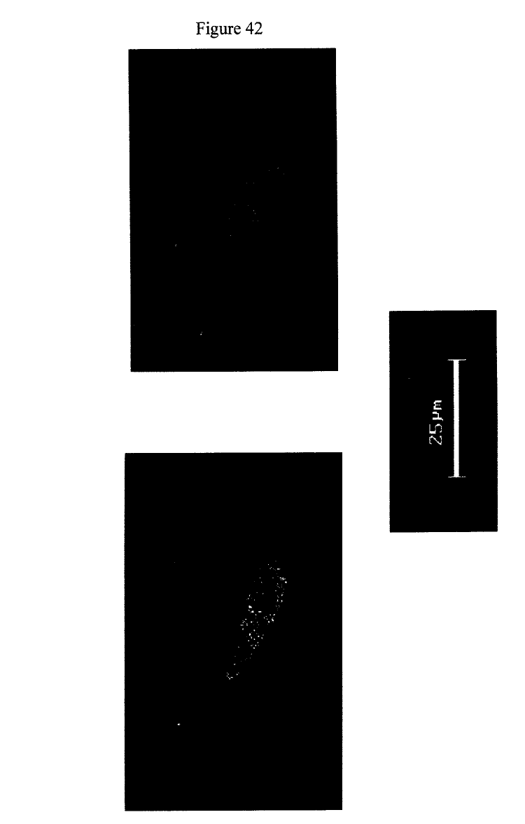

Most of the bacteria-derived crystals studied have shown striking, although variable, auto-fluorescence. With many of the crystals, the red auto-fluorescent has typically been stronger than the green auto-fluorescence. FIG. 40 shows the appearance, through a red filter, of the red auto-fluorescence induced in a bacteria culture derived crystal, using the green laser light source (543 nm). Similar bright red auto-fluorescence was seen with a smaller crystal obtained from a different bacterial culture (FIG. 41). The comparison of green auto-fluorescence (upper panel) and red auto-fluorescence (lower panel) of a small crystal is shown in FIG. 42.

Topic G. Cell Damage Induced in a Stealth Virus Culture Using Light.

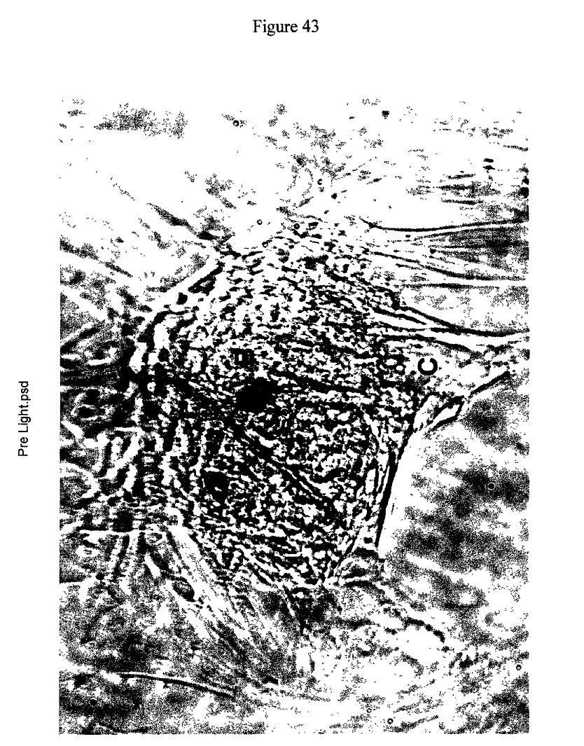

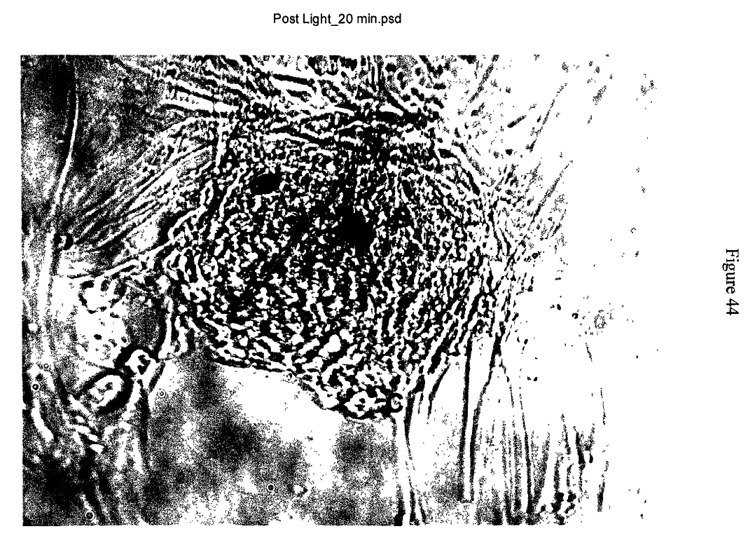

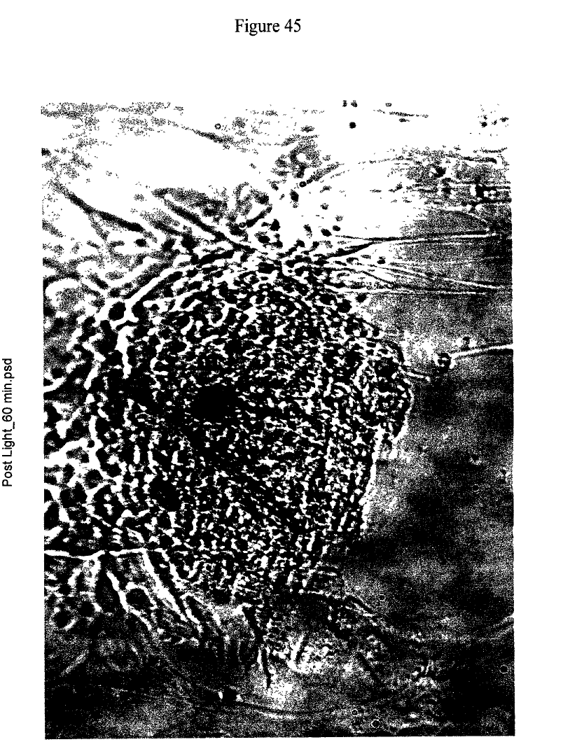

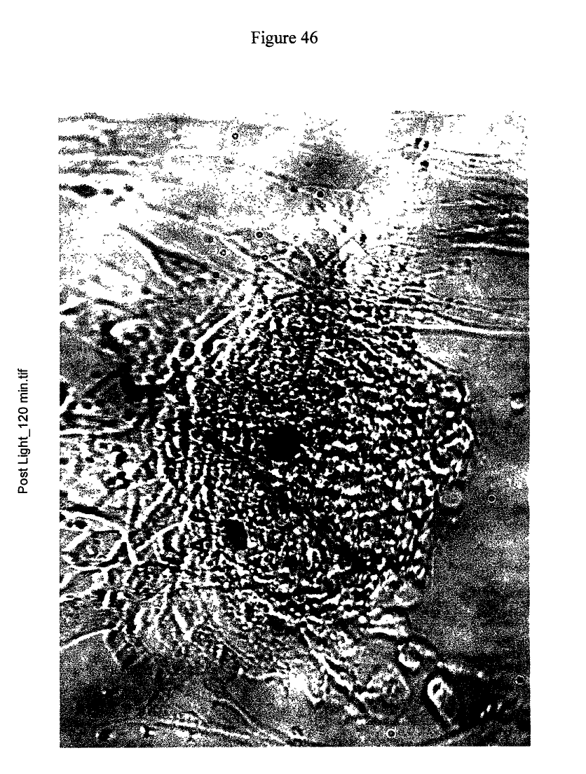

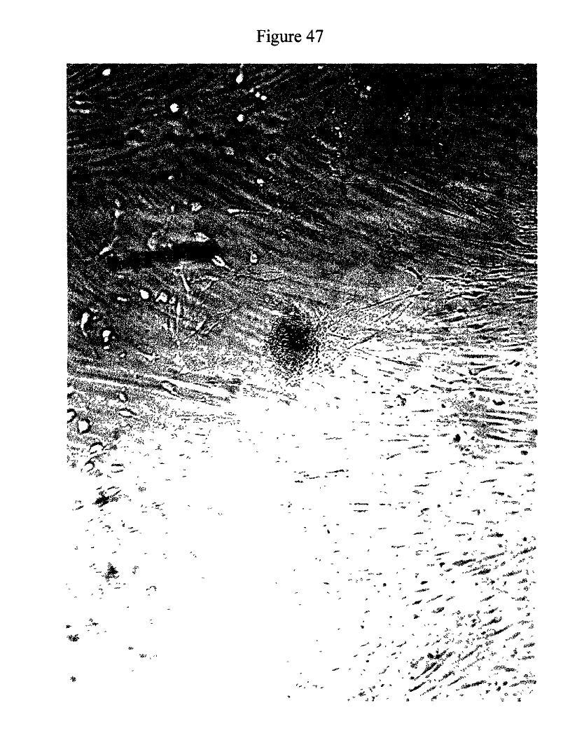

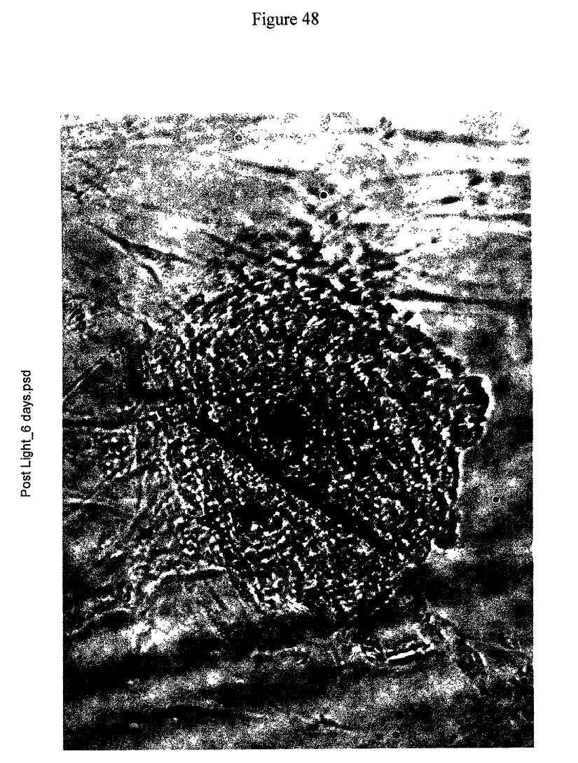

I selected an infrequently refed, largely recovered, stealth virus culture in which a few residual pigmented cell clusters were still present. The pre-light therapy photomicrograph is shown in FIG. 43. It shows the capacity of recovering cells to localize some of the previously produced pigmented intracellular materials, in this case into two clearly defined deposits labeled A and B. It also shows the capacity of the cluster of cells to synthesize a thin tubular extraneous structure extending through the cell cluster. Prior to light therapy, relatively normal appearing cells were seen extending from the lower right side of the cell cluster (labeled C). These cells began to withdraw within 20 minutes of light exposure from the microscope (FIG. 44), and the change was readily apparent at 60 (FIG. 45) and at 120 minutes (FIG. 46) of light exposure. There was also an overall increase and broader redistribution of the pigmented material. Other nearby regions of previously healthy looking cells also showed considerable cell damage that progressed over the next several hours. A low power photograph of the area surrounding the cell cluster that was taken 4 hours after terminating the bright light exposure, is shown in FIG. 47. What was even more striking in this and other similar experiments, was the lack, even over the next 21 days, of any subsequent recovery of cell viability. A photograph taken at day 6 is provided as FIG. 48. It shows the lack of any viable cells emerging from the cell cluster. The lack of a healing response is in striking contrast with the rapidity of the repair that normally ensures after activating a culture by replacing the tissue culture medium. In these cultures, the recovery process is generally clearly apparent within 12-24 hours after activation has occurred. In several other studies, the time required to cause cell damage has been reduced to 10 minutes exposure with evidence of a subsequent loss of viability after the illumination had been discontinued. Control cultures consisting of normal fibroblasts showed no signs of cell damage induced by exposures of from 2-15 hours to light from the same microscope.

I can, therefore, conclude that light is selectively toxic for cells from certain stealth virus cultures. The toxicity can be ascribed to the formation of intracellular auto-fluorescent materials in many of the stealth virus cultures examined. The discovery of this material, along with the finding of light induced cellular toxicity of stealth virus infected cells, has opened up an exciting treatment option for stealth virus infected patients, including animals. Early clinical studies using light in a patient with breast cancer have provided encouraging results. These and other studies will be continued upon submission of this specification.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Additional advantages and modifications will readily occur to those skilled in the art. Variations and changes may be made without departing from the spirit of the invention

What I claim as my invention is:

1. A method to test for the ability of a stealth virus to induce the production of auto-fluorescent material in cells, comprising i) culturing blood and/or tissue samples from a stealth virus infected patient in a manner that will allow for the expression of a stealth virus induced cytopathic effect, ii) illuminating the cells and/or cell-derived products with light, and iii) examining the infected cells and/or cell derived products, microscopically for the presence of auto-fluorescent material.

2. A method to test for the ability of a stealth virus to induce the production of auto-fluorescent material in bacteria, comprising i) either culturing bacteria derived from a stealth virus infected patient, or culturing normal bacteria that have been exposed to a stealth virus culture, ii) illuminating the cultured bacteria and/or their products with light, and iii) examining the bacteria and/or their products microscopically for the presence of auto-fluorescent material.

3. A method of testing the ability of light to cause damage to stealth virus infected cells that have accumulated auto-fluorescent material, by exposing the cells to light that is capable of inducing auto-fluorescence in the infected cells, and then examining the cells for morphological and/or functional signs of cell damage, that may include cell death.

4. A method of treating a stealth virus infected patient based on exposing known or suspected virus infected cells within the patient to a light source that includes wavelengths that can be shown to cause auto-fluorescence and morphological and/or functional signs of cell damage to cultured cells infected with a stealth virus obtained from the infected patient, and not to cause similar morphological and/or functional signs of cell damage to corresponding normal uninfected cells.

5. The method of claim 4 in which the stealth virus infected cells are causing diseases in animals.

6. The method of claim 4 in which the stealth virus infected cells in the patient are present within a cancer.

7. The method of claim 5 in which the therapy is applied to a known cancer mass present within the individual to be treated.

8. The method of claim 5 in which the therapy is applied to any area of the body in which one might expect or suspect microscopic deposits of cancer cells to be present.

9. The method of claim 4 in which the stealth virus infected cells are causing a nonmalignant illness in a patient.

10. The method of claim 4 in which the exposure to light therapy is restricted to part of the body, or to an external device, through which the patients blood is flowing, such that over time, most of the entire circulating blood volume would be expected to be exposed to the administered light.

11. A method of treating a stealth virus infected patient based on exposing stealth virus infected bacteria within the patient to a light source that includes wavelengths shown to cause auto-fluorescence of the stealth virus infected bacteria and/or of the products derived from the infected bacteria.

12. The method of claim 9 in which the stealth virus infected bacteria are causing a bacterial illness.

13. A method of destroying all or some of any suspected stealth virus infected cells in a blood sample, or in a blood unit to be used for transfusion, or in other biological samples, comprising the use of light administered to the sample in such a manner that has previously been shown to cause the destruction of at least a proportion of the stealth virus infected cells in other blood samples and/or blood units that were known to be infected with a stealth virus.

14. A method of claim 8 in which the stealth virus infected cells are suspected of being present in a tissue graft to be used for transplantation.

15. A method of destroying all or some of any suspected stealth virus infected bacteria in an environmental sample, comprising the use of light administered to the sample in such a way that it can be shown to cause the destruction of at least a proportion of the stealth virus infected bacteria in a comparable environmental sample that was known to be infected with a stealth virus.

16. A method to test for the ability of a stealth virus to induce the production of material in cells that can be activated by an energy source other than light, comprising i) culturing blood and/or tissue samples from a stealth virus infected patient in a manner that will allow for the expression of a stealth virus induced cytopathic effect, ii) exposing the cells and/or cell-derived products to the energy source, and iii) examining the infected cells for morphological and/or functional signs of cell damage, that may include cell death.

17. The method of claim 15, in which the energy source is provided by any of the following modalities used alone or in combination with one another: radio-frequency radiation, magnetic field radiation or ultrasound vibration.

18. The method of claim 15, in which the energy source is used together with light to achieve the desired effect of causing damage to a stealth virus infected cell.

19. A method of treating a stealth virus infected patient based on exposing virus infected cells within the patient to a form of energy, other than light, that can be shown to cause morphological and/or functional signs of cellular damage to cultured cells infected with the stealth virus derived from the patient and not to cause similar morphological and/or functional signs of cell damage to corresponding normal uninfected cells.

20. The method of claim 18, in which the energy source is provided by any of the following modalities used alone or in combination with one another: radio-frequency radiation, magnetic field radiation, or ultrasound vibration.

21. The method of claim 18, in which the energy source is used together with light to achieve the desired effect of causing damage to the stealth virus infected cells within a patient.
